(19) United States
(12) Patent Application Publication
(10) Pub. No.: US 2026/0027240 A1
(43) Pub. Date: Jan. 29, 2026

(54) FLUORESCENCE REPORTER AND/OR THERAPEUTIC SYSTEMS, DEVICES, AND METHODS OF USE

(71) Applicant: MolecuLight Inc., Toronto (CA)

(72) Inventor: Ralph S. DACOSTA, Etobicoke (CA)

(73) Assignee: MolecuLight Inc., Toronto, ON (CA)

(21) Appl. No.: 18/864,757

(22) PCT Filed: May 9, 2023
(86) PCT No.: PCT/CA2023/050627

§ 371 Date: Nov. 11, 2024

Related U.S. Application Data
(60) Provisional application No. 63/340,158, filed on May 10, 2022.

(51) Int. Cl. A61K 49/00

(52) U.S. Cl. CPC A61K 49/0073

(57) ABSTRACT

Fluorescence reporting systems and methods useful in characterizing substrates are provided. The system can be in the form of a device such as a bandage for a wound or packaging for a consumable. The system can comprise a fluorophore and a quencher configured to quench the fluorophore. It also can comprise an absorption matrix configured to receive a primary agent, for example, an enzyme, from a substrate. The primary agent can alter a proximity of the quencher to the fluorophore to affect energy transfer between them. The device can be applied to and removed from the substrate. Fluorescence or other radiation emitted by the device or system can be monitored and detected. The presence or absence of fluorescence can indicate a characteristic of the substrate, for example, a bacterial infection.

### TECHNICAL FIELD

The present disclosure relates to systems configured for, devices equipped for, and methods of characterizing a substrate, for example, a wound or other tissue comprising object, using reflectance, absorption, and/or fluorescence activated by or emitting in one or more wavelengths, wherein the wavelengths are from any one of or any combination of ultraviolet (UV), visible, near-infrared (NIR), and infrared (IR) wavelength ranges.

### Introduction

Wounds manifest themselves in a wide range of patient populations. Although wounds can be the result of a myriad of different causes from sudden traumatic injury to more gradual causes such as prolonged bedrest (e.g. pressure injuries) and metabolic conditions, one of their main commonalities and issues is accurate characterization at the time of (diagnostic) assessment. It is important to determine if a wound is infected and how the wound is progressing over time, including whether the patient is responding to treatments. The inability to identify wound that are infected can lead to delayed intervention and this contributes to the chronicity of wounds. Traditionally, the diagnosis of wound infections has been characterized by sight alone, which provides relatively little information regarding the number and types of cells, bacterial burden or important diagnostic biological markers present within the wound bed or surrounding tissues. In some instances, wounds can be characterized by elevated temperature compared with surrounding healthy tissues and this is often subjectively characterized by touching the wound. Improved characterization of wounds would help a clinician better understand whether a wound is colonized or infected by bacteria (compared with traditional methods), intervene earlier with treatment and better identify which treatments would have the greatest efficacy for a particular wound. In addition, the ability to better characterize wounds could lead to accelerated healing.

Wounds are just one example of a substrate that can be subject to intrusion by bacteria and other microorganisms with potentially adverse consequences. Other such substrates include packaged foods, for example, fruits, vegetables, dairy products, meat, poultry, and fish, which are susceptible to spoilage that can result in undesirable flavor profiles and potentially illness to those that consume them. Other substrates include ex vivo tissues such as organs as well as blood or sera intended for use in patients. What all these substrates have in common is that removing the bandage, packaging, or other container can compromise the sterility and stability of the substrate. Traditionally removal has been required to test for contamination, yet the removal can lead to the very contamination that one is trying to avoid. Thus, there is a need for less disruptive methods for characterizing the sterility, health, or other features of substrates.

### SUMMARY

In accordance with an example of the present disclosure, a fluorescence reporting system is provided. The system can comprise a fluorophore and a quencher configured to quench the fluorophore. The system also can comprise an absorption matrix configured to receive a primary agent from a substrate. The system can further comprise a barrier that can assume a permeable state and an impermeable state. The primary agent can convert the barrier from the impermeable state to the permeable state. The barrier can be impermeable to the fluorophore and the quencher in the impermeable state. The system can comprise a container separated from the absorption matrix by the barrier. The barrier can form part of the container, help define the extend of the container, or both. The absorption matrix can comprise the fluorophore and the container can comprise the quencher in the impermeable state. Alternatively, the absorption matrix can comprise the quencher and the container can comprise the fluorophore in the impermeable state. In the permeable state, the container can contain both the fluorophore and the quencher, the absorption matrix can comprise both the fluorophore and the quencher or both. The system can be a device, for example, a bandage or dressing. The system can comprise the device and further components, for example, an optical radiation source and an optical radiation detector.

In accordance with another example of the present disclosure, a fluorescence reporting system is provided. The system can comprise a fluorophore, a linker, a quencher, and an absorption matrix. The quencher can be configured to quench the fluorophore in a quenching state. The linker can be configured to maintain the quenching state. The absorption matrix can comprise the fluorophore, the quencher, and the linker. The absorption matrix can be configured to receive a primary agent from a substrate. The primary agent can be configured to establish a non-quenching state. The system can be a device, for example, a bandage or dressing. The system can comprise the device and further components, for example, an optical radiation source and an optical radiation detector.

In accordance with another example of the present disclosure, a fluorescence reporting system is provided. The system can comprise a fluorophore, a quencher, and an absorption matrix. The quencher can be configured to quench the fluorophore in a quenching state. The absorption matrix can comprise the fluorophore and the quencher. The absorption matrix can be configured to receive a primary agent from a substrate. The primary agent can be configured to establish the quenching state by decreasing an average distance between the fluorophore and the quencher. The system can be a device, for example, a bandage or dressing. The system can comprise the device and further components, for example, an optical radiation source and an optical radiation detector.

In accordance with another example of the present disclosure, a method of characterizing a substrate is provided. The method can comprise one or more of the following steps. A device on, or previously adjacent to, a substrate can be exposed to radiation at a wavelength configured to excite a fluorophore. The device can comprise, for example, any system described herein. For example, the device can comprise an absorption matrix, the fluorophore, and a quencher. The quencher can be configured to quench the fluorophore. The absorption matrix can be configured to receive a primary agent from the substrate. The primary agent can be configured to affect the quenching of the fluorophore by the quencher. Fluorescence from the fluorophore or the quencher or both can be monitored. A presence or an absence of the primary agent in the substrate can be identified based on the fluorescence monitoring. The method can comprise applying the device to the substrate, removing the device from the substrate, or both.

Additional objects and advantages of the present disclosure will be set forth in part in the description which follows, and in part will be obvious from the description, or can be learned by practice of the present disclosure. The objects and advantages of the present disclosure will be realized and attained by means of the elements and combinations particularly pointed out in the appended claims.

It is to be understood that both the foregoing general description and the following detailed description are exemplary and explanatory only and are not restrictive of the present disclosure, as claimed.

The accompanying drawings, which are incorporated in and constitute a part of this specification, illustrate one (several) embodiment(s) of the present disclosure and together with the description, serve to explain the principles of the present disclosure.

### BRIEF DESCRIPTION OF DRAWINGS

In accordance with the present disclosure, the following figures are provided.

FIG. 1A depicts a side schematic view of a system before application to a substrate with the barrier in an impermeable state with fluorophores in an absorption matrix separated from quenchers in a container.

FIG. 1B depicts the system in FIG. 1A after application to the substrate and before absorption of a primary agent from the substrate.

FIG. 1C depicts the system after absorbing the primary agent.

FIG. 1D depicts the primary agent affecting the barrier of the system.

FIG. 1E depicts the barrier in a permeable state after being affected by the primary agent.

FIG. 1F depicts the fluorophores interacting with the quenchers after crossing the barrier in the permeable state.

FIG. 1G is a plan view corresponding to the system depicted in FIGS. 1A-1D in which graphic indicia are visible but not highlighted.

FIG. 1H is a plan view corresponding to the system depicted in FIG. 1F in which the graphic indicia are highlighted by fluorescence emitted from the system.

FIG. 2A depicts a side schematic view of a system before application to a substrate with the barrier in an impermeable state with quenchers in an absorption matrix separated from fluorophores in a container.

FIG. 2B depicts the system in FIG. 2A after application to the substrate and before absorption of a primary agent from the substrate.

FIG. 2C depicts the system after absorbing the primary agent.

FIG. 2D depicts the primary agent affecting the barrier of the system.

FIG. 2E depicts the barrier in a permeable state after being affected by the primary agent.

FIG. 2F depicts the quenchers interacting with the fluorophores after crossing the barrier in the permeable state.

FIG. 3A depicts a side schematic view of a system comprising vesicles in an absorption matrix before application to a substrate with the vesicles in an impermeable state with fluorophores in the absorption matrix separated from quenchers in the vesicles.

FIG. 3B depicts the system in FIG. 3A after application to the substrate and before absorption of a primary agent from the substrate.

FIG. 3C depicts the system after absorbing the primary agent.

FIG. 3D depicts the primary agent affecting the vesicles of the system.

FIG. 3E depicts the vesicles in a permeable state after being affected by the primary agent.

FIG. 3F depicts the fluorophores interacting with the quenchers after entering the vesicles in the permeable state.

FIG. 4A depicts a system comprising a housing before the system is applied to a substrate.

FIG. 4B depicts the system in FIG. 4A after application to a substrate.

FIG. 5A depicts a side schematic view of a system after application to a substrate with the barrier in an impermeable state with fluorophores in an absorption matrix separated from quenchers and a substrate-affecting agent in a container.

FIG. 5B depicts the system in FIG. 5A after the primary agent has converted the barrier from the impermeable state to the permeable state allowing the fluorophore to enter the container and the surface-affecting agent to leave the container and reach the substrate through the absorption matrix.

FIG. 6A depicts a side schematic view of a system before application to a substrate with a barrier in an impermeable state with fluorophores in an absorption matrix separated from quenchers in a container, and secondary agents tethered in the absorption matrix to prevent them from affecting the barrier.

FIG. 6B depicts the system in FIG. 6A after application to the substrate and before absorption of a primary agent from the substrate.

FIG. 6C depicts the system after absorbing the primary agent with the primary agent affecting the tethers.

FIG. 6D depicts the system after the secondary agent has been released by the primary agent.

FIG. 6E depicts the barrier in an impermeable state being affected by the secondary agent.

FIG. 6F depicts the fluorophores interacting with the quenchers after crossing the barrier in the permeable state.

FIG. 7A depicts a side schematic view of a system before application to a substrate with vesicles in an impermeable state containing secondary agents to prevent them from affecting the barrier.

FIG. 7B depicts the system in FIG. 7A after application to the substrate and before absorption of a primary agent from the substrate.

FIG. 7C depicts the system after absorbing the primary agent with the primary agent affecting the vesicles.

FIG. 7D depicts the system after the secondary agent has been released from the vesicles by the primary agent.

FIG. 7E depicts the barrier in an impermeable state being affected by the secondary agent.

FIG. 7F depicts the fluorophores interacting with the quenchers after crossing the barrier in the permeable state.

FIG. 8 depicts a side schematic view of a system before application to a substrate with the barrier in an impermeable state with first and second fluorophores in an absorption matrix separated from first and second quenchers in a container.

FIG. 9 depicts a side schematic view of a system before application to a substrate with the barrier in an impermeable state with first and second quenchers in an absorption matrix separated from first and second fluorophores in a container.

FIG. 10A depicts a side schematic view of a system before application to a substrate with the barrier in an impermeable state with first and second fluorophores in respective chambers of an absorption matrix separated from first and second quenchers in respective compartments of a container.

FIG. 10B depicts the system in FIG. 10A after application to the substrate and before absorption of first and second primary agents from the substrate.

FIG. 10C depicts the system after absorbing the first and second primary agents.

FIG. 10D depicts the first and second primary agents affecting respective first and second regions of the barrier of the system.

FIG. 10E depicts the barrier in a permeable state after being affected by the first and second primary agent.

FIG. 10F depicts the first and second fluorophores interacting with the first and second quenchers after crossing the barrier into respective compartments of the container in the permeable state.

FIG. 10G is a plan view corresponding to the system depicted in FIGS. 10A-10D in which the first and second graphic indicia are visible but not highlighted.

FIG. 10H is a plan view of the system in which the first graphic indicia are highlighted indicating the presence of the first primary agent.

FIG. 10I is a plan view of the system in which the second graphic indicia are highlighted indicating the presence of the second primary agent.

FIG. 10J is a plan view corresponding to the system depicted in FIG. 10F in which both the first and second graphic indicia are highlighted by fluorescence emitted from the system.

FIG. 11A depicts a side schematic view of a system before application to a substrate with fluorophores bound to quenchers through respective linkers, the linkers permitting the fluorophores and quenchers to achieve sufficiently proximity for energy transfer to occur.

FIG. 11B depicts the system in FIG. 11A after application to the substrate and before absorption of a primary agent from the substrate.

FIG. 11C depicts the system after the primary agent has been absorbed from the substrate.

FIG. 11D depicts the system after the primary agent has bound to the linkers.

FIG. 11E depicts the system after the primary agent has cleaved the linkers.

FIG. 11F depicts the system after the previously linked fluorophores and quenchers have dispersed and no longer having a sufficiently close proximity to one another for energy transfer to occur.

FIG. 12A depicts a side schematic view of a system with fluorophores bound to quenchers through respective linkers, the linkers permitting the fluorophores and quenchers to achieve sufficiently close proximity for energy transfer in a first configuration, after application of the system to a substrate and absorption of primary agents from the substrate, the primary agents beginning to affect the linkers.

FIG. 12B depicts the system after the primary agent has converted the linkers to a second configuration such that the fluorophores and quenchers are no longer sufficiently close for energy transfer to occur.

FIG. 13A depicts a side schematic view of a system before application to a substrate with fluorophores bound to quenchers through respective linkers, the linkers permitting the fluorophores and quenchers to achieve sufficiently proximity for energy transfer in a first configuration maintained by a secondary agent.

FIG. 13B depicts the system in FIG. 13A after application to the substrate and after absorption of a primary agent from the substrate.

FIG. 13C depicts the system after the primary agent has bound to the secondary agent.

FIG. 13D depicts the system after the primary agent has converted the linkers to a second configuration by removing the secondary agent from the linkers such that the fluorophores and quenchers are no longer sufficiently close for energy transfer to occur.

FIG. 14A depicts a side schematic view of a system before application to a substrate with fluorophores and quenchers freely dispersed in an absorption matrix without sufficient proximity to one another for energy transfer to occur.

FIG. 14B depicts the system in FIG. 14A after application to the substrate and before absorption of a primary agent from the substrate.

FIG. 14C depicts the system after the primary agent has been absorbed from the substrate.

FIG. 14D depicts the system after the primary agent has bound the fluorophores and quenchers permitting the fluorophores and quenchers to achieve sufficiently proximity for energy transfer to occur.

FIG. 15 is a flow chart depicting steps of a method for characterizing the status of a substrate.

FIG. 16 is a side schematic view of a system including a multispectral imager.

FIG. 17 is a bar graph depicting a series of readouts.

### DETAILED DESCRIPTION

The present disclosure provides systems, devices, and methods for characterizing substrates that are covered, enclosed, or otherwise inaccessible to direct characterization. Fluorescent reporters in or on the cover or other enclosure allow the substrate to be characterized without the removal of the cover or enclosure. The substrate may be any portion of an object for which the possible presence of bacterial, viral, fungal, or other microorganismal infection or contamination is a concern. For example, the substrate may be, without limitation, a wound, a lesion, an abscess, a surgical or post-surgical site (e.g., an attachment point for a colostomy bag), an incision point (e.g. an insertion point for a peripherally inserted central catheter (PICC) line), a treatment device that provides physical access into a patient body (where that access could provide ingress for bacteria or other contaminants) such as a port, portion of a PICC line, urinary catheter, surgical drain, medical implant, etc. The substrate need not be limited to medical environments and can also encompass other areas in which bacterial or other contamination may be a concern or danger to either the user of the product or the process in which the product is being used, such as food safety (e.g., a food wrapping) or forensic or laboratory settings (e.g., gloves worn by investigator or clinician). In the case of a wound, the system can provide an in situ alternative to a biopsy. The fluorescent reporters comprise fluorophores and compatible quenchers whose proximity to one another is controlled directly or indirectly by an agent obtained from the substrate. This agent can be, for example, a biomarker such as a molecule from a bacterial or host cell in the substrate this is absorbed by the covering. The molecule can be specific to or otherwise representative of a given type and/or state of a bacterial, host, or other cell. By altering the proximity of a fluorophore relative to a quencher in the covering, the ability of fluorophore to transfer energy to the quencher is altered. The result can be an increase or decrease of fluorescence emitted by the fluorophore, the quencher, or both based on one or more excitation wavelengths. The change in fluorescence emitted can be detected by an in situ or external detector. The bacterial, host or other cell can then be identified or narrowed based on change. In the case of a bandage or dressing, knowing the cell type or status can help in diagnosis of the wound state and to identify appropriate treatment. That diagnosis can aid a patient or health professional in determining whether the wound is healing or if further therapy should be administered, if the patient should seek prompt medical attention (e.g. for the early identification of infection that would not have otherwise been apparent to the patient or health professional), and so on. For items such as catheters, PICC lines, and surgical drains, the identification of bacteria in an area that provides ingress to the body (via a port, insertion, incision or other body opening) can indicate early infection or potential danger that would recommend replacement of the catheter, PICC line, or drain before infection sets in or becomes worse. Often, these types of medical devices are not replaced on a daily basis or replacement is reserved for when infection has become apparent to the naked eye, which may be a point at which the infection has become advanced. In the case of a food wrapping, knowing the type or status of a cell, bacteria, enzyme, etc. can help determine if the wrapped food is safe to eat or should be discarded, and may inform quality control during food preparation and/or storage. The prompt identification of contamination on gloves used in clinical, forensic, and/or laboratory settings can reduce or avoid contamination of the wearer's work.

While the following detailed description provides examples in the form of fluorescence reporters, the present disclosure is not so limited. In practice, the present disclosure may be implemented in the form of an optical reporter that operates using the principles of bioluminescence, phosphorescence, optical absorption, and/or reflectance in addition to or instead of the principle of fluorescence.

While the following detailed description is primarily directed to an exemplary implementation using a fluorophore and a quencher configured to quench the fluorophore, the present disclosure is not limited to this particular mechanism of fluorescence emission activation, imaging, or spectroscopic readouts. In other exemplary implementations, the present disclosure may be implemented for fluorescence lifetime imaging or spectroscopy; enzyme-activated fluorescent dyes, which can be read out by imaging or spectroscopic means; antibody or Fab-targeted fluorescent dyes, which bind to target ligands on bacteria, tissues, cells, or other soluble biological components of wounds; color-based (e.g., RGB) imaging and spectroscopy; Raman-emitting molecules or Fluorescence Resonance Energy Transfer (FRET)-based molecular dyes; photoacoustic imaging, optical coherence tomography; and/or scattering dyes/molecules that enhance reflectance of excitation light, thereby to increase contrast between the target and the background.

As used herein, a “biomarker” may include, without limitation, matrix metalloproteinases (MMPs), tissue inhibitors of metalloproteinases (TIMPs), bacterial Gram sign, stem cell indicators, serum or plasma indicators, platelets, blood, growth factors, immune factors, exosomes, pH indicators, cytokines, polysaccharides (e.g. in biofilms), oxygen indicators, nitric oxide indicators, reactive oxygen species indicators, keratinocytes, fibroblasts, endothelial cells, other enzymes, and the like. A biomarker may be any cellular, bacterial, or other component whose paracrine action includes secreting various cytokines and growth factors including but not limited to fibroblast growth factor, vascular endothelial growth factor (VEGF), hepatocyte growth factor, interleukin (IL)-6, IL-8, granulocyte colony-stimulating factor, platelet-derived growth factor AA (PDGF-AA), and/or granulocyte-macrophage colony-stimulating factor.

In accordance with the present disclosure, a fluorescence reporting system, such as an intelligent optical wound dressing, is provided. The system can be considered, for example, a device or a device in combination with other components. The system can comprise a fluorophore and a quencher configured to quench the fluorophore. The system also can comprise an absorption matrix configured to receive a primary agent from a substrate. The system can further comprise a barrier that can assume a permeable state and an impermeable state. The primary agent can convert the barrier from the impermeable state to the permeable state. The barrier can be impermeable to the fluorophore and the quencher in the impermeable state. The system can comprise a container separated from the absorption matrix by the barrier. The barrier can form part of the container, help define the extent of the container, or both. The absorption matrix is so called as it is designed to be placed adjacent or proximal the substrate for absorbing the primary agent. But the container can also be absorptive in nature. The matrix, solvent, other base components of the absorption matrix and container can be the same, similar, or different.

The devices, systems, and methods described herein may be used as a stand-alone or may be integrated into a larger system. In one example, the fluorescent reporting system described herein may provide information that is combined with other wound data (e.g., bacterial autofluorescence or load, wound size/depth, texture, tissue classification, temperature and so on) to determine whether a wound is infected and/or to determine the healing rate or status of a wound. To assist with such determinations, the fluorescent reporting system described herein may be used to make measurements over time (e.g., over multiple visits to a healthcare facility) to document changes in any wound or healthy tissue parameters.

The absorption matrix can comprise the fluorophore and the container can comprise the quencher in the impermeable state. Alternatively, the absorption matrix can comprise the quencher and the container can comprise the fluorophore in the impermeable state. In the permeable state, the container can contain both the fluorophore and the quencher, the absorption matrix can comprise both the fluorophore and the quencher or both. Permeable and impermeable states are labeled with respect to the permeability of the barrier to the primary agent. For example, in the impermeable state, while impermeable to the primary agent, the barrier can be permeable to other molecules. Similarly, in the permeable state, while permeable to the primary agent, the barrier can be impermeable to other molecules.

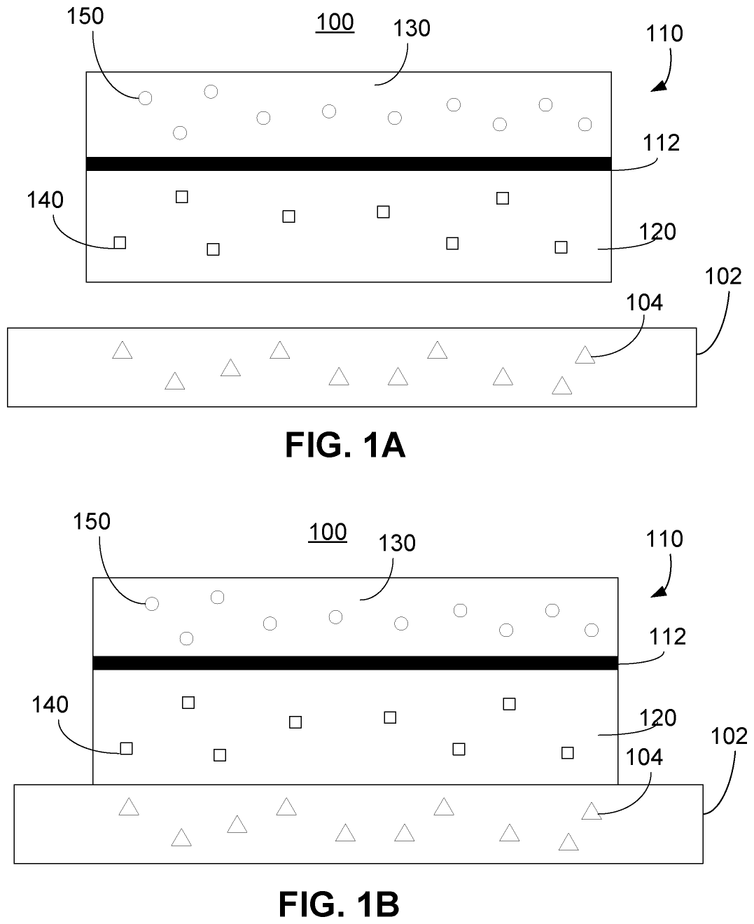

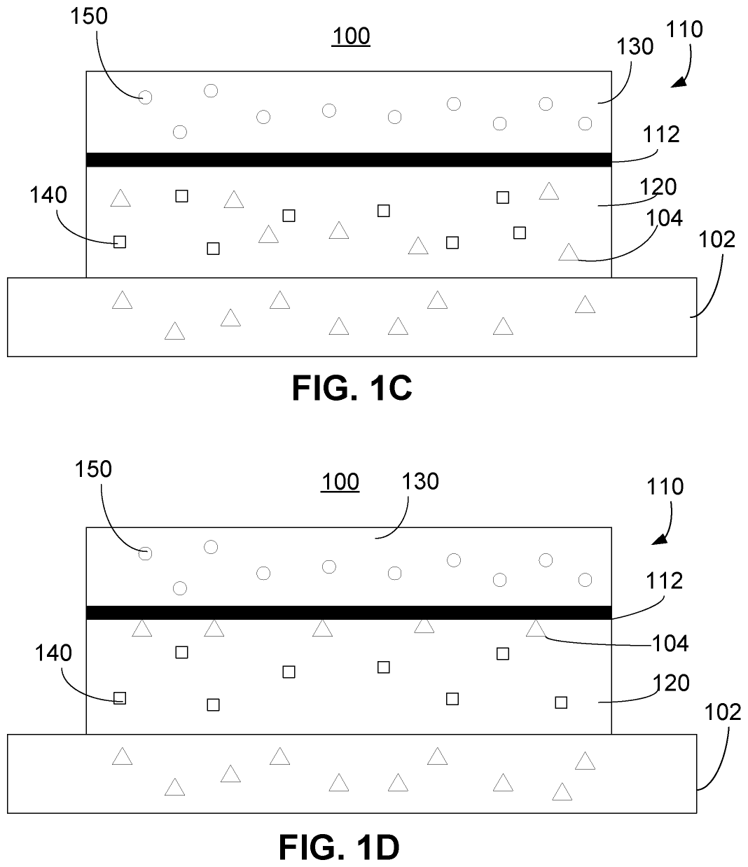

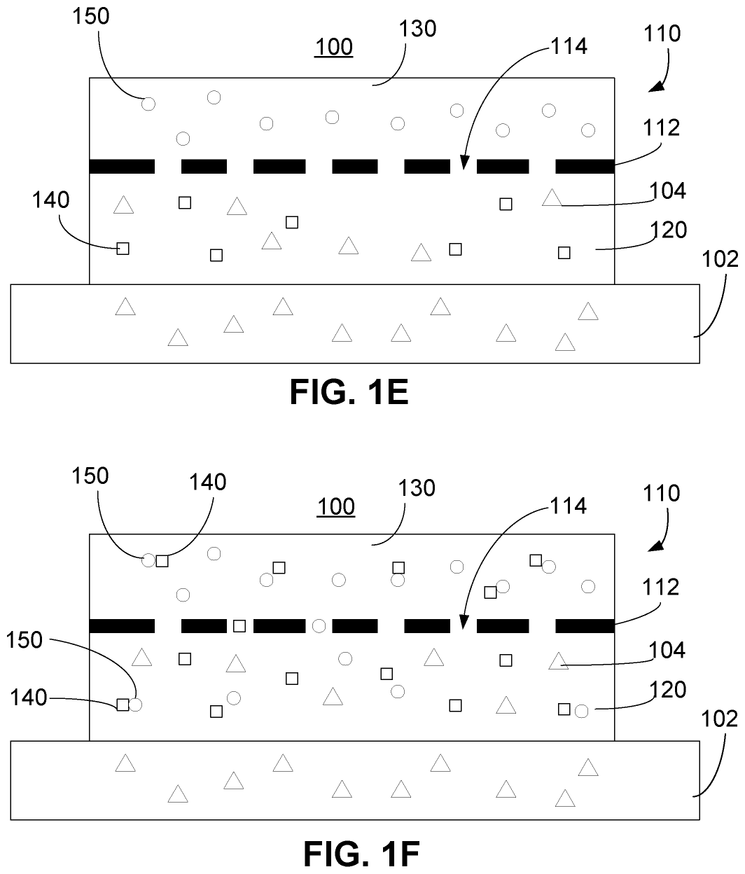

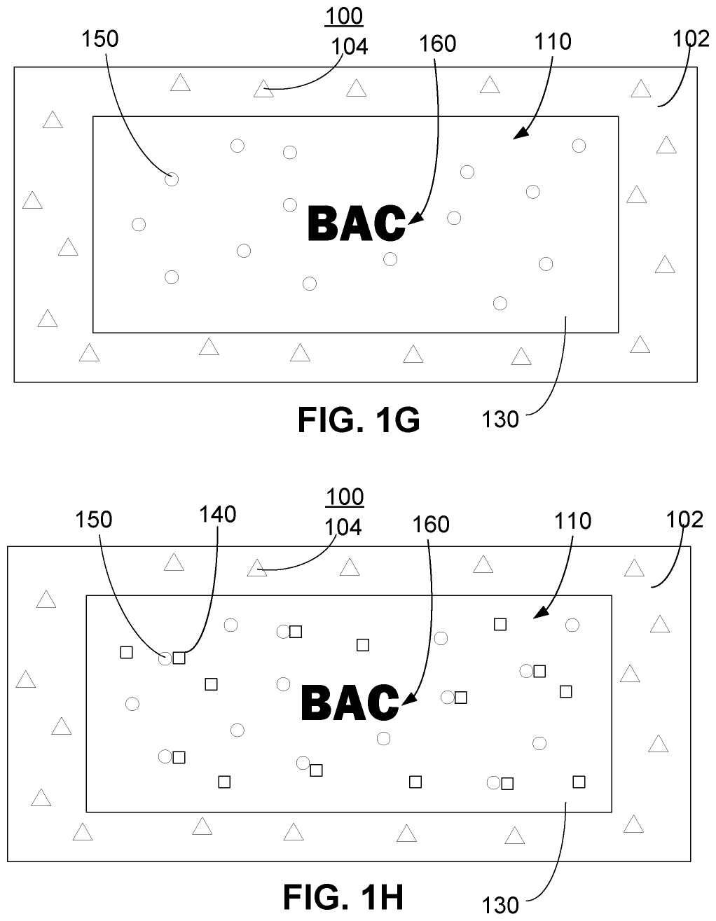

FIG. 1A depicts a side schematic view of a system 100 comprising device 110 before application to a substrate 102 comprising primary agent 104. A barrier 112 in an impermeable state separates fluorophores 140 in an absorption matrix 120 from quenchers 150 in a container 130. The views are conceptual in nature as at least the fluorophores and quenchers themselves would generally be microscopic. The system and system components are also not drawn to scale or proportional relative to one another. Device 110 can be considerably thinner than the substrate, for example as a bandage on a wound of a patient's limb, but it is shown larger to allow for clear depiction of its various components.

FIG. 1B depicts device 110 in FIG. 1A after application to substrate 102 and before absorption of primary agent 104 from substrate 102. FIG. 1C depicts device 110 after absorbing primary agent 104. FIG. 1D depicts primary agent 104 affecting barrier 112. FIG. 1E depicts barrier 112 in a permeable state after being affected by primary agent 104. The permeable state is represented by holes 114 in barrier 112. But, the permeable state need not comprise actual holes or other openings in the barrier. The permeable state can be achieved by selectively changing the permeability of barrier 112 through other mechanisms, for example, a change in pH, a change in surface tension, a change in the hydrophilic/lipophilic balance, a change in polarity, a molecular gate, or any combination thereof. In the permeable state, barrier can remain essentially intact or disintegrate in whole or part.

FIG. 1F depicts fluorophores 140 interacting with the quenchers 150 after crossing barrier 112 in the permeable state. This interaction can result in an increase, decrease, or both in fluorescence with respect to fluorophore, the quencher, or both. For example, the interaction can comprise a direct or indirect transfer of energy resulting in a change in fluorescence of the fluorophore, the quencher, or both. Although FIG. 1F shows fluorophore 140 entering and present in container 130, as well as quencher 150 entering absorption matrix 120, the disclosure also encompasses embodiments in which only one occurs. For example, in the permeable state, barrier 112 can be permeable to fluorophore 140, but impermeable to quencher 150, or vice versa. Permeability can also be configured to be direction specific. For example, the permeability can be configured such that fluorophore 140 can enter container 130, but not be able to pass back across barrier 112 into absorption matrix 120.

Fluorescence can be used as a reporter alone or in combination with one or more other features, for example, graphic indicia (features). The graphic indicia may be or include words, phrases, symbols, brand names, colors, and so on. FIG. 1G is a plan view corresponding to device 110 depicted in FIGS. 1A-1D in which graphic indicia 160 are visible but not highlighted. FIG. 1H is a plan view corresponding to device 110 depicted in FIG. 1F in which the graphic indicia 160 are highlighted by fluorescence emitted from device 110 as a result of energy transfer from fluorophores 140 to quenchers 150. In other implementations, at least some of the graphic indicia 160 may initially be invisible and may become visible as a result of energy transfer from fluorophores 140 to quenchers 150. Moreover, the graphic indicia 160 may initially be partly visible and partly invisible and become fully visible as a result of the energy transfer; for example, to change from a minus sign to a plus sign. These are examples, and the disclosure also encompasses embodiments wherein a decrease or disappearance of fluorescence and associated lack of highlighting can be informative of the status of the underlying substrate.

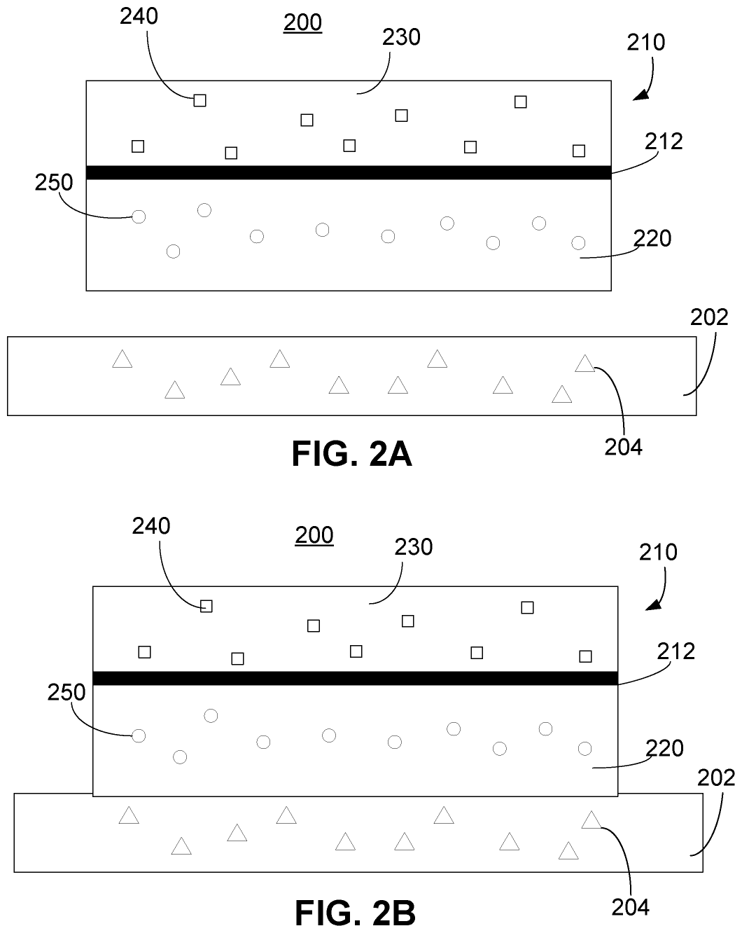

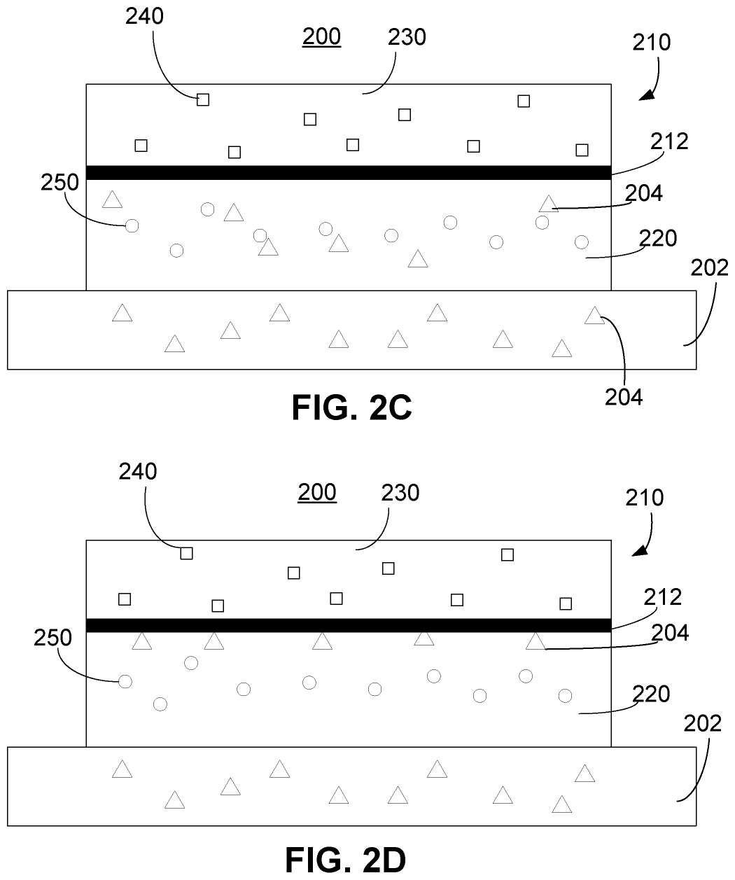

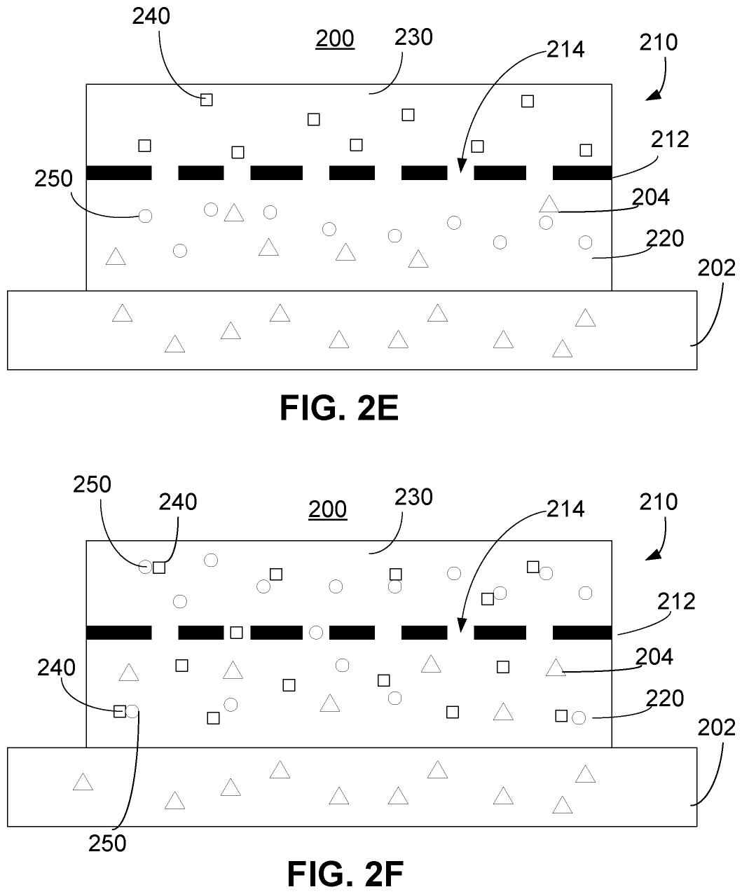

FIG. 2A depicts a side schematic view of a system 200 comprising a device 210 before application to a substrate 202 comprising primary agent 204. A barrier 212 in an impermeable state separates quenchers 250 in an absorption matrix 220 from fluorophores 240 in a container 230. Thus, FIG. 2A, depicts an embodiment in which the starting location of fluorophores and quenchers is the reverse of what is shown in FIG. 1A. FIG. 2B depicts device 210 in FIG. 2A after application to substrate 202 and before absorption of primary agent 204 from substrate 202. FIG. 2C depicts device 210 after absorbing primary agent 204. FIG. 2D depicts primary agent 204 affecting barrier 212. FIG. 2E depicts barrier 212 in a permeable state after being affected by the primary agent. Holes 214 represent the permeable state. FIG. 2F depicts quenchers 250 interacting with fluorophores 240 after crossing barrier 212 in the permeable state.

While the above description of FIGS. 1A-2F is presented with only a single species of primary agent 104 or 204, barrier 112 or 212, fluorophore 140 or 240, and quencher 150 or 250, respectively, in some implementations a plurality of different species may be provided. For example, the device 110 or 210 may include multiple active areas laterally displaced from one another, each corresponding to a different wound biomarker (e.g., matrix metalloproteinases or MMPs, or other enzymes, tissue inhibitors of metalloproteinases or TIMPs, bacterial Gram sign, stem cell indicators, serum or plasma indicators, platelets, blood, growth factors, pH indicators, polysaccharides (e.g. in biofilms), oxygen indicators, nitric oxide indicators, reactive oxygen species indicators, and/or other biomarkers described above) and each with a unique fluorescence emission wavelength. Concentrations and relative levels of each biomarker can be read out and analyzed by, for example, an artificial intelligence (AI) algorithm and measured over time.

Moreover, while the above description of FIGS. 1A-2F is presented with regard to a fluorophore 140 or 240 and a quencher 150 or 250, in some implementations another optical reporting mechanism, such as phosphorescence and/or luminescence, may be provided in addition to or instead of fluorescence. In one particular example, the device 110 or 210 may utilize bioluminescence which may eliminate the need for an excitation light source in order to read the device 110 or 210. In such implementations, some (e.g., only in certain lateral areas) or all of the fluorophore 140 or 240 may be replaced with a bioluminescence imaging (BLI) probe and some (e.g., only in certain lateral areas) or all of the quencher 150 or 250 may be replaced with a luciferase, or vice versa. The BLI probe and luciferase may be separated by the barrier 112 or 212 in the form an enzyme-digestible membrane. When the primary agent 104 or 204 (e.g., a bacterial enzyme) is expressed, the barrier 112 or 212 is made at least partially permeable, resulting in the mixing of the BLI probe and luciferase to generate visible light that can be seen with the naked eye.

In still other implementations, the device 110 or 210 may include a therapeutic implementation in which at least a portion of the container 130 or 230 is impregnated with a therapeutic. The therapeutic may be initially prevented from contacting the substrate 102 or 202, for example by barrier 112 or 212. When the primary agent 104 or 204 (e.g., a molecule that is indicative of a bacterial, viral, fungal, or other microorganismal infection) is expressed, the barrier 112 or 212 is made at least partially permeable. As a result, the therapeutic may migrate toward the substrate 102 or 202 and thereby treat or otherwise alleviate the condition (e.g. the infection) which caused the primary agent 104 or 204 to be expressed. In one particular example, the therapeutic may be an antibiotic or other drug treatment. As noted above, in certain implementations multiple species of the primary agent 104 or 204 may be present. In such implementations, the therapeutic may be configured to treat or otherwise alleviate the condition which caused one or more, but not all, species of the primary agent 104 or 204 to be expressed. For example, the primary agent 104 or 204 may include both metalloprotease 1 (MMP1) and metalloprotease 9 (MMP9). The therapeutic may prove a partial treatment through the release of only a tissue inhibitor of metalloproteinase 1 (TIMP1) or a tissue inhibitor of metalloproteinase 9 (TIMP9) but not both. Therefore, the remaining species of the primary agent 104 or 204 may remain present in order to cause the optical effect being measured (e.g. fluorescence, luminescence, phosphorescence, absorption, reflection, etc.).

In implementations where a fluorophore and quencher are present, the fluorophore can fluoresce, or the quencher can fluoresce, or both. The fluorophore can absorb incident radiation to move from a low energy state to a high energy state. The fluorophore can emit radiation or otherwise transfer energy to move back to the low energy state or another energy state. The fluorophore can transfer energy directly or indirectly to a quencher. For example, the fluorophore can transfer energy to the quencher via FRET. The quencher can emit radiation or otherwise transfer energy to another molecule. In embodiments in which the quencher is not fluorescent, the quencher can serve to extinguish the fluorescence of the fluorophore, or transfer energy to another molecule that does fluoresce, or both. The quenching of the fluorophore by the quencher can result in a change in emitted radiation in the system. The change in emitted radiation can comprise a change in color. The radiation emitted by the fluorophore, the quencher, or both can be detected by sight alone or by using one or more instruments.

Any suitable camera or other detector can be used to detect fluorescence or other radiation from the device. For example, the camera may be the image sensor of a mobile communication device such as a smartphone, and/or may be a peripheral attachment designed for use with the mobile communication device. In another example, a camera equipped with a fluorescence filter, including a multispectral fluorescence imaging device, can be used. For example, the MolecuLight i:X imaging device available from MolecuLight Inc. of Toronto, Ontario, Canada, which includes a 5 megapixel camera, emits at 405 nm, and includes fluorescence emission filters of 500-545 nm and 600-665 nm respectively, may be used. In another example, the MolecuLight i:X and DX imaging devices available from MolecuLight Inc. of Toronto, Ontario, Canada, which include a fluorescence camera, at least one white-light camera, multiple fluorescence light-emitting diodes (LEDs) emitting at 405 nm±15 nm, a white-light LED and fluorescence emission filters which transmit light of 500-545 nm and 600-665 and block light of >700 nm, may be used. An imaging device and associated methods described in U.S. Pat. No. 9,042,967, which is incorporated by reference in its entirety, can be used. Alternatively, one of the devices disclosed in U.S. Provisional Patent Application No. 62/625,967, filed Feb. 2, 2018 and entitled “Devices, Systems, and Methods for Tumor Visualization and Removal,” and in U.S. Provisional Patent Application No. 62/625,983, filed Feb. 3, 2018 and entitled “Devices, Systems, and Methods for Tumor Visualization and Removal,” the entire content of each of which is incorporated herein by reference, may be used to capture white-light images and/or fluorescence images.

The camera or other detector (e.g., whether a smartphone or other mobile communication device is used, or a multispectral fluorescence imaging device is used) may be equipped with a processor and a memory to implement the systems and methods described herein. The camera or other detector may include a non-transitory computer readable medium storing instructions (e.g., a program) that, when executed by the processor, cause the camera or other detector to perform one or more of the methods, subroutines, or actions described herein. The camera or other detector may further include a communication interface to communicate data and/or commands to an external device and/or to receive data and/or commands from the external device. The communication interface may implement a wired communication protocol, including but not limited to Universal Serial Bus (USB), IEEE 1394 (FireWire), Thunderbolt, optical fiber, or Ethernet protocols. The communication interface may additionally or alternatively implement a wireless communication protocol, including but not limited to Wi-Fi, Bluetooth, Zigbee, near field communication (NFC), or cellular communication protocols. In one example, the communication interface may be configured to provide the automatic reporting of data acquired by the system or device, for example to a clinician. The camera or other detector may be configured to operate in any optical wavelength including any one or any combination of UV, visible, NIR, and IR wavelengths.

The absorption matrix can comprise any suitable material or combination of materials. For example, the absorption matrix can comprise a hydrogel. The hydrogel can be a biocompatible hydrogel. The composition of the absorption matrix can be tailored to favor the absorption of one or more primary agents of interest. The composition of the absorption matrix can be tailored to disfavor the absorption of one or more other substances. The absorption matrix may be transparent, semi-transparent, or opaque. The absorption matrix may include any one or any combination of sheet, amorphous gel, or sheet hydrogel-impregnated dressings, including those made of agar, gelatine, or materials obtained from the mixture of natural polymers (e.g. agar-agar, gelatine, pectin) and synthetic polymers (e.g. poly(vinyl pyrrolidone)) (PVP), poly(ethylene glycol) (PEG), etc.); tissue substitutes; placental tissues; dressings made of woven or non-woven materials in a variety of shapes and sizes; transparent films; hydrocolloids; aliginates; composites; and other types of wound dressings.

The barrier can comprise any suitable material or combination of materials. For example, the barrier can comprise a membrane. The membrane can be a natural membrane, a synthetic membrane, or both. The barrier can be formulated and calibrated to adjust its susceptibility to particular primary agents. For example, the barrier can be configured to reflect in a particular amount or range of primary agent present in the substrate. The barrier may be transparent, semi-transparent, or opaque. The barrier may include any one or any combination of polymer membranes (natural and/or synthetic), amniotic membranes, protein membranes, cellular membranes, connective tissue membranes (e.g., keratin, collagen, elastin, etc.), hydrogel, foam, alginate, hydrocolloid, fiber, or film membranes, silk, cellulose membranes, lipid membranes, plastic membranes, paper membranes, electrospun membranes, silver membranes, nanotechnology membranes, carbon fiber membranes, electrically conductive membranes, carbon nanotube membranes, graphene membranes, nanofiber meshes, microfiber meshes, and the like.

Any suitable fluorophore or quencher can be used. The fluorophore may be, for example and without limitation, one or a combination of fluorescent dyes such as Cascade Blue, Alexa Fluor 405, AMCA-X, Alexa Fluor 350, Pacific Blue, Marina Blue, Epoch Blue, ATTO 390, ATTO 425, EDANS, BODIPY 493/503, ATTO465, Cy2, BODIPY FL, Alexa Fluor 488, FAM, DY-495, ATTO 488, HiLyte Fluor 488, fluorescein, Oregon Green 488-X, Oregon Green 488, Oregon Green 514, ATTO 495, Oyster 500, Rhodamine Green, Rhodamine Green-X, DY-505, Alexa Fluor 430, TET, ATTO 520, Lucifer Yellow, Alexa Fluor 514, CAL Fluor Gold 540, Yakima Yellow, AP5, BODIPY R6G, ATTO 532, Alexa Fluor 532, BODIPY 530/550, VIC, JOE, HEX, CAL Fluor Orange 506, DY-485XL, Alexa Fluor 555, HiLyte Fluor 555, Quasar 570, BODIPY 558/568, Cy3, Oyster-556, BODIPY 564/570, DY-554, DY-555, DY-556, Alexa Fluor 546, BODIPY TMR, NED, Dragonfly Orange, ATTO 550, DY-560, Rhodamine Red-X, TAMRA, Rhodamine Red, DY-510XL, BODIPY 576/589, ATTO 565, CAL Fluor Red 590, BODIPY 581/591, PET, Cy3.5, DY-590, ROX, Alexa Fluor 568, CAL Fluor Red 610, LC Red 610, Texas Red, HiLyte Fluor 594, BODIPY TR, Alexa Fluor 594, ATTO 590, ATTO 594, Alexa Fluor 610, DY-610, DY-480XL, ATTO 610, CAL Fluor Red 635, BODIPY 630/650, LC Red 640, DY-615, ATTO 620, Alexa Fluor 633, PULSAR 650, ATTO 633, DY-630, DY-631, DY-632, DY-633, BODIPY 650/665, DY-601XL, DY-520-XL, ATTO 647N, Alexa Fluor 647, Oyster-645, Cy5, Quasar 670, WellRED D4-PA, DY-635, DY-636, DY-647P1, DY-650, Oyster-656, HiLyte Fluor 647, DY-651, ATTO 655, Alexa Fluor 660, Cy5.5, IRDye 700, ATTO680, DY-675, DY-676, Alexa Fluor 680, Quasar 705, WellRED D3-PA, DY-680, DY-681, LC Red 705, ATTO 700, Alexa Fluor 700, DY-700, DY-701, ATTO 725, DY-730, DY-731, DY-732, ATTO 740, WellRED D2-PA, Alexa Fluor 750, Cy7, DY-750, HiLyte Fluor 750, DY-751, DY-776, DY-780, DY-781, DY-782, or IRDye 800. The fluorophore may additionally or alternatively include oxygen sensing dyes, reactive oxygen sensing dyes, phosphorescent dyes, semiconductor quantum dotes The quencher may be, for example and without limitation, one or a combination of DDQI, DABCYL, BHQ-0, QXL520, EDQ, BHQ-1, QXL570, BHQ-2, QXL610, DDQII, QXL670, BHQ-3, or IR-QXL.

The present disclosure may also implement fluorescent thermometry, for example by using a fluorescent dye which exhibits substantial temperature dependence, thereby to infer the temperature of a fluid or surface. In such an implementation, the dye may be dissolved in a fluid of interest or coated on a surface, and then caused to fluoresce by incident excitation light. The fluoresced light may then be recorded (e.g. with a camera) and variations in the intensity are proportional to variations in the local temperature. Changes to a temperature of a wound can indicate a change in status of the wound. For example, an increase in temperature or elevated temperature (above a predetermined threshold level) may be indicative of infection in a wound or surgical site. Similarly, a decrease in temperature or low temperature (below a predetermined threshold level) may be indicative of blocked/reduced blood flow or a clot at, for example, a surgical site, or possibly even of necrotic tissue.

Any suitable material or combination of materials can be used for the container. The material composition of the container can be the same or different than that of the absorption matrix, and may include one or more of the materials listed above.

The system can comprise a first solution comprising the fluorophore and a second solution comprising the quencher. The absorption matrix can comprise the first solution and the container can comprise the second solution in the impermeable state. Alternatively, the absorption matrix can comprise the second solution and the container can comprise the first solution in the impermeable state. The first and second solutions can be fully or partially miscible such that mixing can occur between them in the permeable state. The first and/or second solutions may be chemically incorporated to the absorption matrix materials. The solutions or solvents may include phosphate-buffered saline (PBS), hydrogels, or any of the other materials listed above in liquid, semi-liquid, or solid/permeable states.

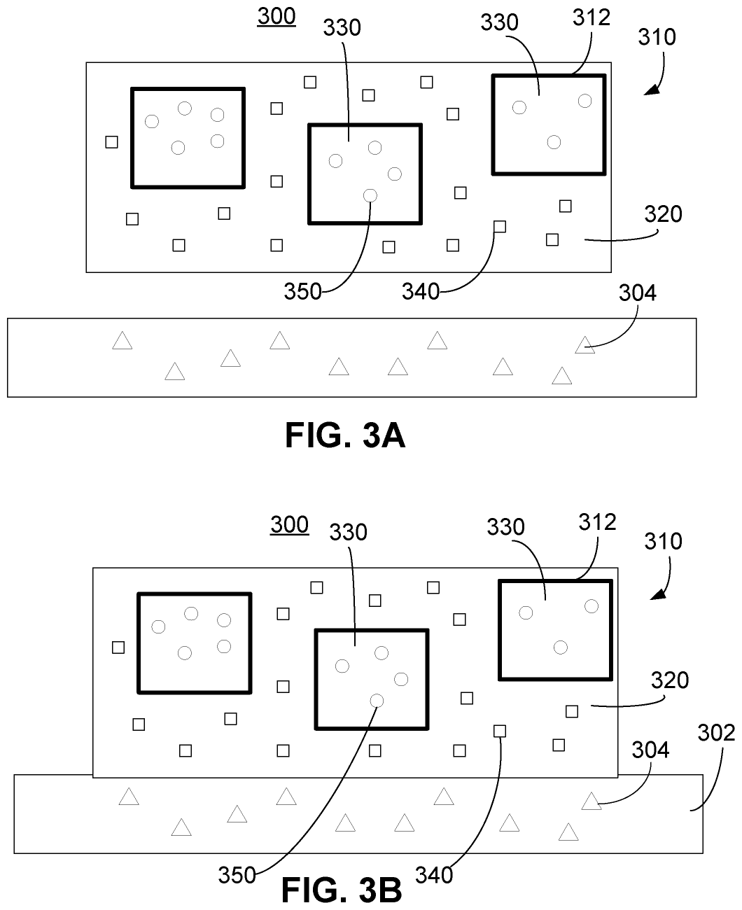

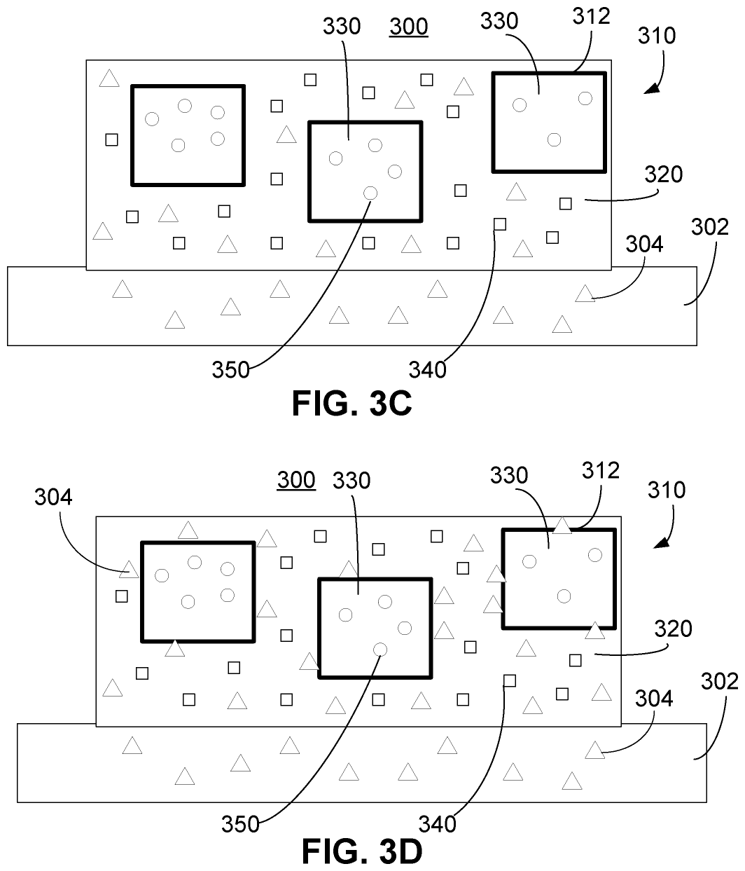

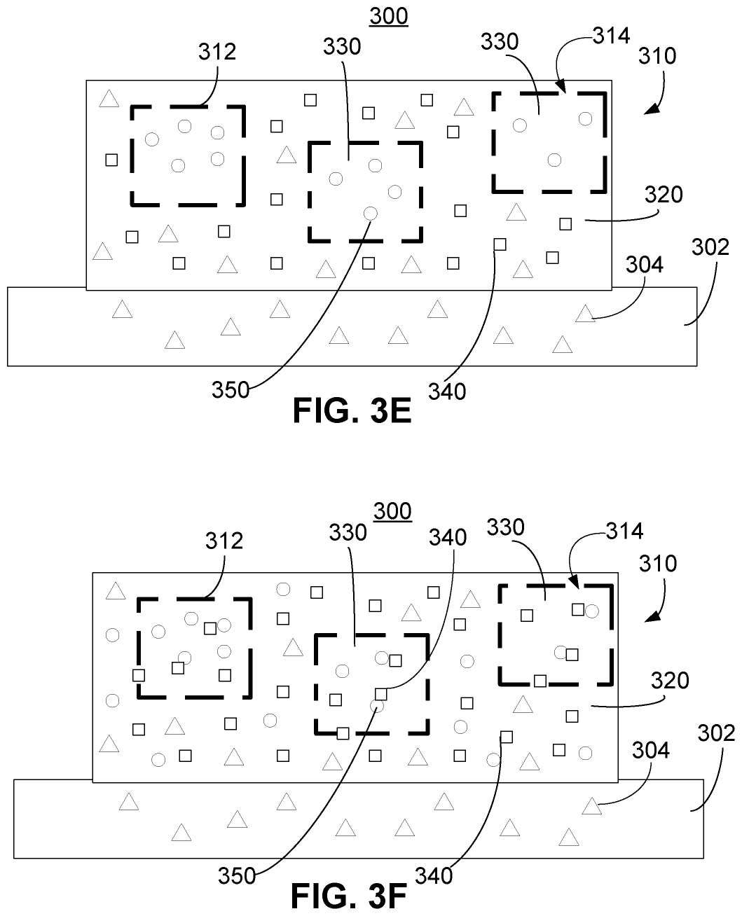

The system can comprise a first layer comprising the absorption matrix, a second layer comprising the barrier, and a third layer comprising the container. The second layer can be between the first and third layers. The first layer can be configured for placement on a substrate to permit absorption of the primary agent from the substrate to the absorption matrix. Examples of layered embodiments are shown in FIGS. 1A-2F. As an alternative to or in addition to a layered configuration, the container of the system can be a vesicle surrounded by the absorption matrix. The vesicle can be bound or surrounded by the barrier. The absorption matrix can comprise a plurality of containers. An example of a vesicle embodiment is shown in FIGS. 3A-3F.

FIG. 3A depicts a side schematic view of a system 300 comprising device 310. Device 310 comprises vesicles 330 defined by a barrier 312 in an absorption matrix 320 before application to a substrate 302 comprising primary agent 304. Vesicles 330 in an impermeable state separate fluorophores 340 in absorption matrix 320 from quenchers 350 in vesicles 330. FIG. 3B depicts device 310 in FIG. 3A after application to substrate 302 and before absorption of primary agent 304 from substrate 302. FIG. 3C depicts device 310 after absorbing primary agent 304. FIG. 3D depicts primary agent 304 affecting vesicles 330. FIG. 3E depicts vesicles 330 in a permeable state after being affected by primary agent 304. Holes 314 represent the permeable state. FIG. 3F depicts fluorophores 340 interacting with the quenchers 350 after entering vesicles 330 in the permeable state. The permeable state of the vesicles can comprise disintegration of the vesicles. That is, the vesicles can disintegrate with the quenchers being released rather than the fluorophores actually entering the vesicles. Embodiments in which the quenchers 350 are in absorption matrix 320 and fluorophores 340 in vesicles 330 initially are also part of the present disclosure.

While the above description of FIGS. 3A-3F is presented with only a single species of primary agent 304, barrier 312, fluorophore 340, and quencher 350, respectively, in some implementations a plurality of different species may be provided. For example, the device 310 may include multiple active areas laterally displaced from one another, each corresponding to a different wound biomarker (e.g., MMPs, TIMPs, pH indicators, and/or other biomarkers described above) and each with a unique fluorescence emission wavelength. Concentrations and relative levels of each biomarker can be read out and analyzed by, for example, an AI algorithm and measured over time.

Moreover, while the above description of FIGS. 3A-3F is presented with regard to a fluorophore 340 and a quencher 350, in some implementations another optical reporting mechanism, such as phosphorescence and/or luminescence, may be provided in addition to or instead of fluorescence. In one particular example, the device 310 may utilize bioluminescence which may eliminate the need for an excitation light source in order to read the device 310. In such implementations, some (e.g., only in certain vesicles 330) or all of the fluorophore 310 may be replaced with a bioluminescence imaging (BLI) probe and some (e.g., only in certain vesicles 330) or all of the quencher 350 may be replaced with a luciferase, or vice versa. The BLI probe and luciferase may be separated by the barrier 312 in the form an enzyme-digestible membrane. When the primary agent 304 (e.g., a bacterial enzyme) is expressed, the barrier 312 is made at least partially permeable, resulting in the mixing of the BLI probe and luciferase to generate visible light that can be seen with the naked eye.

In still other implementations, the device 310 may include a therapeutic implementation in which at least a portion of the container 330 (e.g. at least some of the vesicles 330) is impregnated with a surface-affecting agent such as a therapeutic. The therapeutic may be initially prevented from contacting the substrate 302, for example by barrier 312. When the primary agent 304 (e.g., a molecule that is indicative of a bacterial, viral, fungal, or other microorganismal infection) is expressed, the barrier 312 is made at least partially permeable. As a result, the therapeutic may migrate toward the substrate 302 and thereby treat or otherwise alleviate the condition (e.g. the infection) which caused the primary agent 304 to be expressed. In one particular example, the therapeutic may be an antibiotic or other drug treatment. As noted above, in certain implementations multiple species of the primary agent 304 may be present. In such implementations, the therapeutic may be configured to treat or otherwise alleviate the condition which caused one or more, but not all, species of the primary agent 304 to be expressed. For example, the primary agent 304 may include both metalloprotease 1 (MMP1) and metalloprotease 9 (MMP9). The therapeutic may prove a partial treatment through the release of only a tissue inhibitor of metalloproteinase 1 (TIMP1) or a tissue inhibitor of metalloproteinase 9 (TIMP9) but not both. Therefore, the remaining species of the primary agent 304 may remain present in order to cause the optical effect being measured (e.g. fluorescence, luminescence, phosphorescence, absorption, reflection, etc.).

The system can comprise a housing holding one or more of the absorption matrix, the barrier, and the container. The housing can comprise any suitable material or combination of materials, including but not limited to gauze, plastic, polymers, cellulose, skin substitutes, hydrocolloids, hydrogels, cotton, polysaccharides, alginates, transparent films, semitransparent films, composites, foams, silver polyurethanes, polyhexamethylene biguanide, hydrophilic and hydrophobic dressings, monofilament dressings, amorphous gels, honey dressings and so on. The system can further comprise an attachment on the housing configured to attach the housing to the substrate. For example, the attachment can comprise an adhesive, hook and loop mechanism, clasp, buckle, friction fit, vacuum fit, snap fit, or tie, or the like. The housing can be self-sealing, for example, the housing can be configured as a plastic wrap that can be wrapped upon itself. The housing can be flexible. A flexible housing can aid in the attachment to a non-planar substrate, for example, a limb or torso. The housing, and the system generally, can be customized for application to a particular substrate. For example, the housing can be a bandage and the substrate can comprise a wound, the housing can be a food wrapper and the substrate can comprise a food, or the housing can be a bag and the substrate can be blood intended for transfusion or an artificial medical liquid. Regardless of whether a housing is present, the device can be provided in a package, on a temporary backing, or other form of storage. Such packaging can help maintain the sterility of the device and prevent it from drying out.

The housing can have a desired appearance for functional reasons, aesthetics, or both. For example, the housing can be transparent, translucent, or both. A transparent or translucent housing can aid in the detection of fluorescence or other radiation emitted by the system. The housing can comprise a graphic feature (indicia) comprising text, a number, a letter, a symbol, or a pattern, or any combination thereof indicating a state of the system. The graphic feature can comprise a grid, fiducial markers, or both to aid in identification of targets in different area of a substrate, for example, peripheral area relative to central areas of a wound bed. Fluorescence emitted by the system can make the graphic feature more prominent or visible than in the absence of the fluorescence. For example, the graphic feature can be configured to be highlighted by fluorescence from the fluorophore, the quencher, or both. Examples of such embodiments are shown in FIGS. 1G and 1H. Graphic indicia or features can be located in or on any appropriate element of the system.

The system can comprise or be used in conjunction with a complementary system or systems comprising an optical radiation source, an optical radiation detector, or both. Alternatively, or additionally, the housing can comprise an optical radiation source, an optical radiation detector, or both. Accordingly, the housing can comprise a light source configured to excite the fluorophore. Similarly, the housing can comprise a detector configured to detect fluorescence emitted by the fluorophore, fluorescence emitted by the quencher, or both. The housing can further comprise a transmitter to transmit signals or associated data from the detector. The housing can further comprise a receiver connected to the light source configured to activate or otherwise control the light source. A transmitter and a receiver can be configured together as a transceiver. Such components as optical radiation sources, detectors, transmitters and the like can be mounted in or on the same housing as the main device comprising the absorption matrix and container or on separate housings adjacent thereto applied to a common substrate. Unless otherwise specified, this disclosure uses “light” and “radiation” interchangeably.

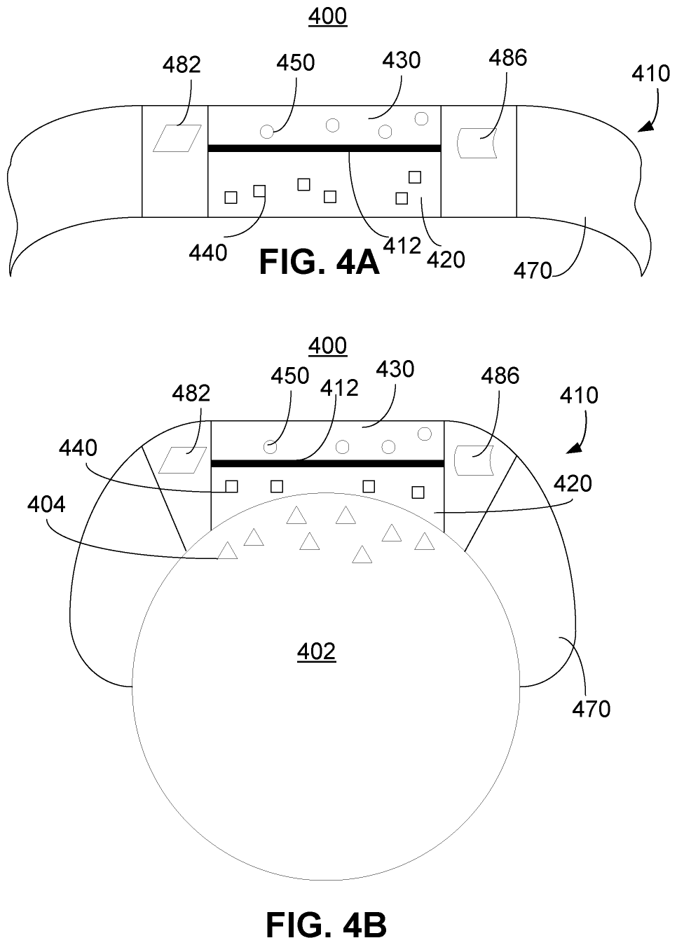

An example of a system comprising a housing is shown in FIG. 4A. FIG. 4A depicts a system 400 comprising device 410. Device 410 comprises a housing 470 before device 410 is applied to a substrate. Apart from the addition of housing 470, device 410 is analogous to device 110 shown in FIG. 1A. Device 410 also comprises a barrier 412 between absorption matrix 420 and container 430. Barrier 412 separates fluorophores 440 from quenchers 450 in the impermeable state. An optical radiation source 482 and an optical radiation detector 486 are also included on housing 470. FIG. 4B depicts device 410 in FIG. 4A after application to a substrate 402. As described above with regard to FIGS. 1A-3F, the device 410 may include multiple active areas laterally displaced from one another, each corresponding to a different wound biomarker and each with a unique fluorescence emission signature. Systems (and/or devices) of the present disclosure can be applied to any appropriate substrate. The substrate can comprise a tissue. The tissue can comprise living tissue, dead tissue, or both. The tissue can comprise tissue of a host or a combination of host tissue and non-host tissue, for example, a xenograph or an allograph. Dead tissue can comprise tissue of the host, for example, scar tissue. A tissue substrate can be in vivo or ex vivo. For example, an in vivo tissue substrate can comprise a wound. An ex vivo tissue can be a medical product, for example, blood. An ex vivo tissue can be a food product.

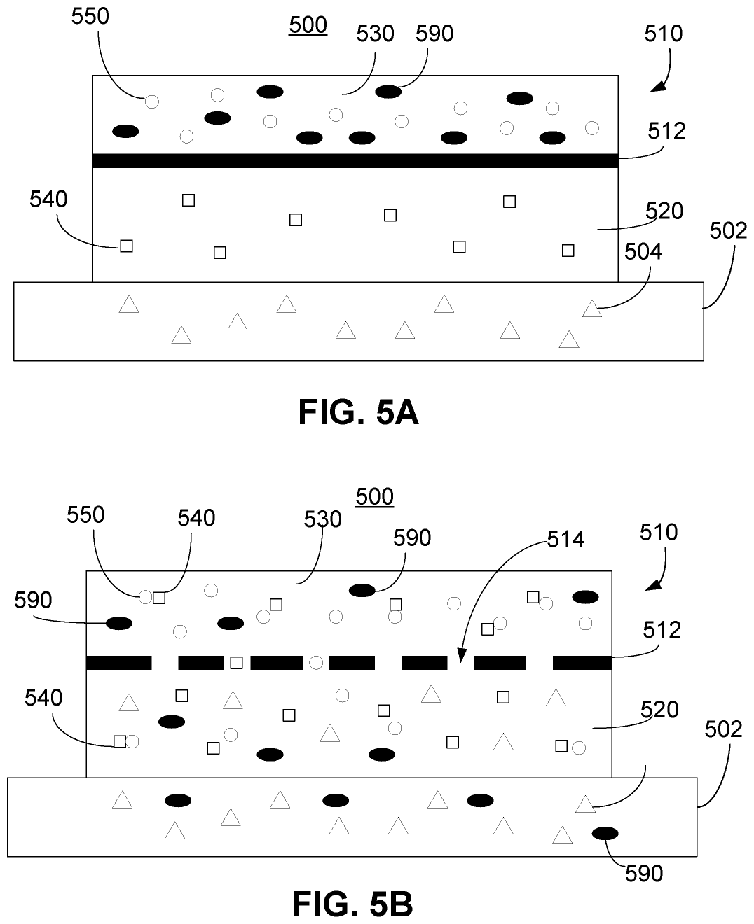

The container can comprise a substrate-affecting agent. The substrate-affecting agent can comprise a therapeutic agent. For example, the therapeutic agent can comprise an antibiotic, an analgesic, or both. An example of an embodiment comprising a substrate-affecting agent is shown in FIGS. 5A and 5B. FIG. 5A depicts a side schematic view of a system 500 comprising device 510 after application to a substrate 502 comprising primary agent 504. A barrier 512 in an impermeable state separates fluorophores 540 in an absorption matrix 520 from quenchers 550 and a substrate-affecting agent 590 in a container 530. FIG. 5B depicts device 510 in FIG. 5A after primary agent 504 has converted barrier 512 from the impermeable state to the permeable state allowing fluorophores 540 to enter container 530 and surface-affecting agent 590 to leave container 530 and reach substrate 502 through absorption matrix 520. Holes 514 in barrier 513 represent the permeable state. As described above with regard to FIGS. 1A-4B, the device 510 may include multiple active areas laterally displaced from one another, each corresponding to a different wound biomarker and each with a unique fluorescence emission signature.

The primary agent can comprise any substrate derived molecule. The primary agent can act directly, indirectly, or both to convert the barrier from an impermeable to a permeable state. The conversion can be temporary or permanent. The conversion can be reversible or irreversible. The primary agent can comprise, for example, an enzyme. The enzyme and barrier can be configured such that the barrier comprises a substrate acted on by the enzyme to achieve the permeable state. For example, the enzyme can cleave the barrier to achieve the permeable state. Alternatively, an enzyme can modify a substrate in ways other than breaking it apart. For example, an enzyme can chemically modify a substrate by adding or removing a chemical moiety such as a phosphate group, a carbohydrate, or a lipid. The enzyme can be any kind of enzyme. For example, the enzyme can comprise a matrix metalloprotease. An example of a matrix is metalloprotease 7 (MMP7). The enzyme can be of any source. For example, the enzyme can comprise a pathogenic enzyme, a host enzyme, or both. In the case of an enzyme, the primary agent can have a multiplicative effect as a single primary agent can carry out many reactions. Thus, just a few primary agent molecules can have a dramatic effect on the barrier. The enzymes may be or include, for example and without limitation, reactive oxygen species, oxygen, temperature, matrix metalloproteinase, proteases, elastase, thrombin, collagenase, bacterial enzymes, host enzymes, exogenous and endogenous enzymes, serine proteases, pyruvate kinase, proteolytic enzymes, enzymes produced by bacterial biofilms and fungi.

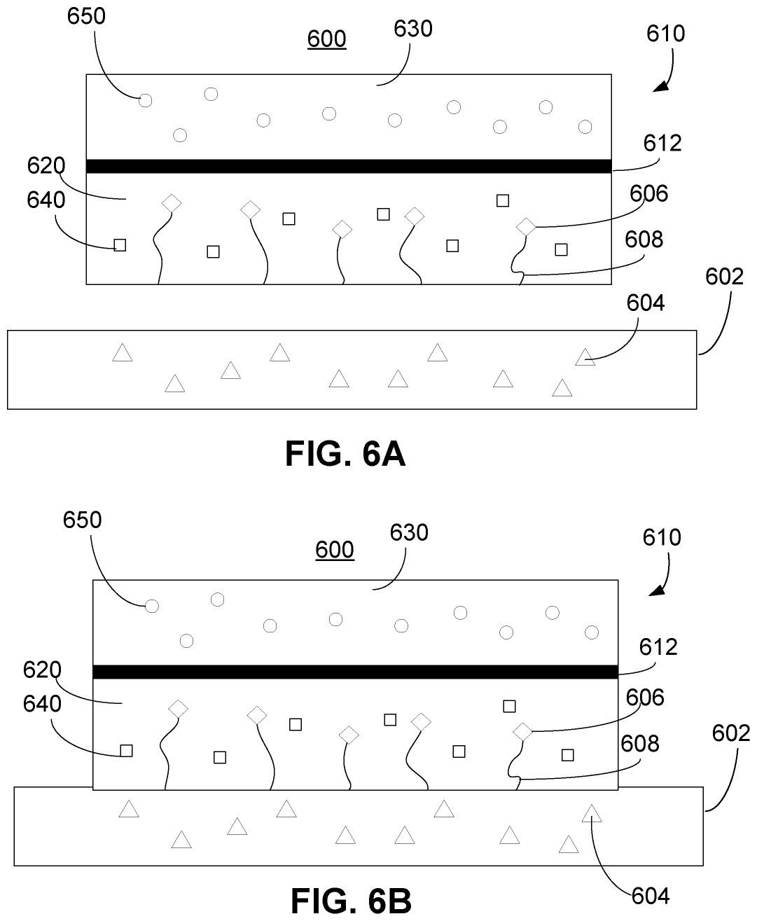

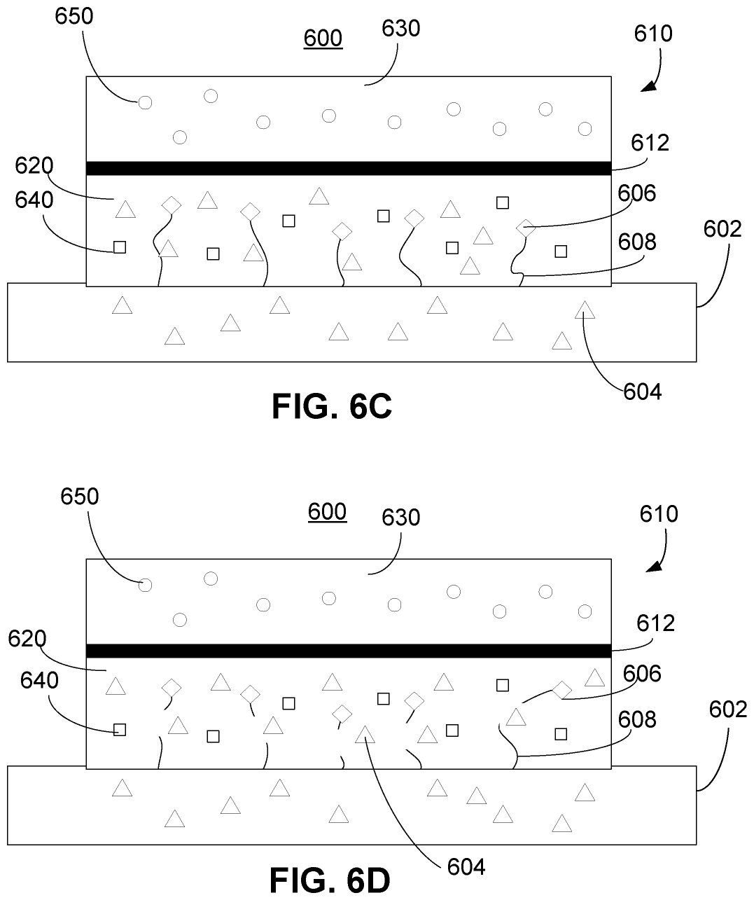

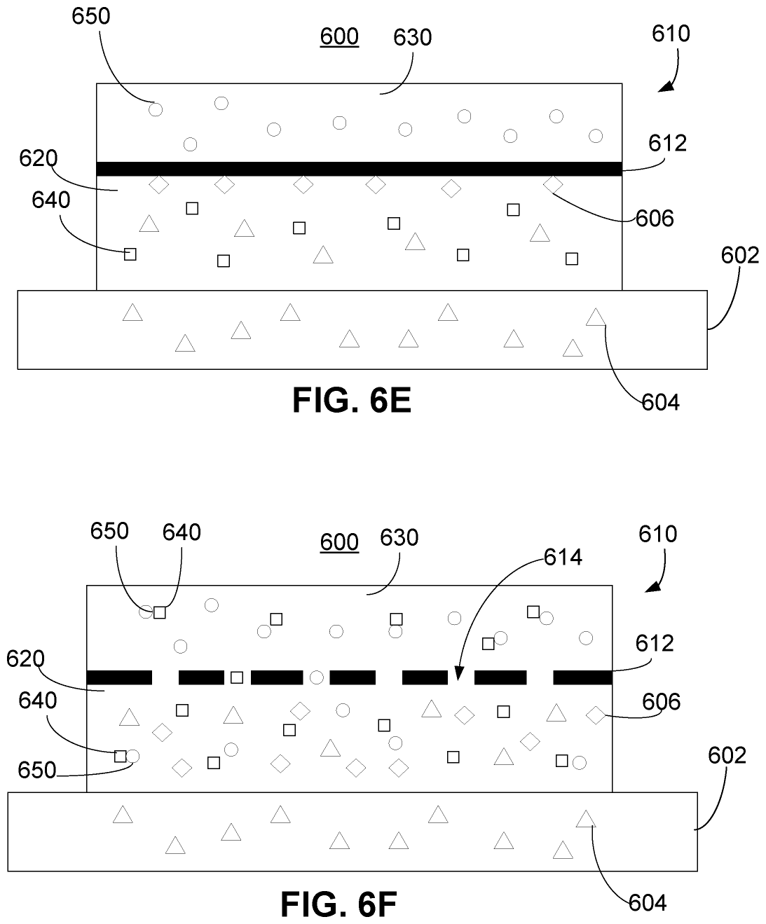

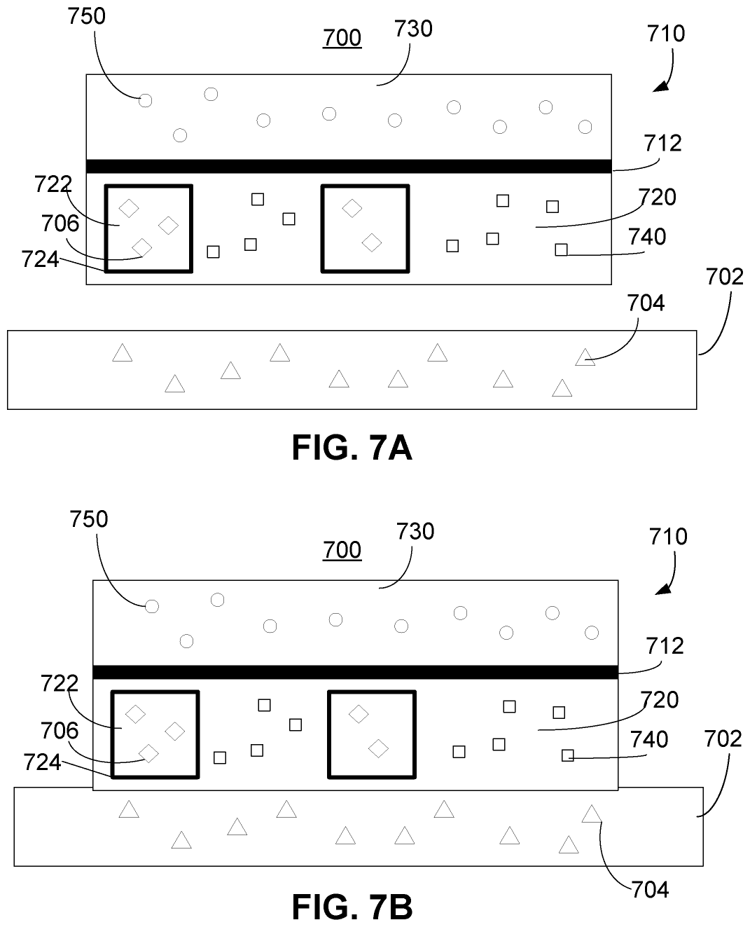

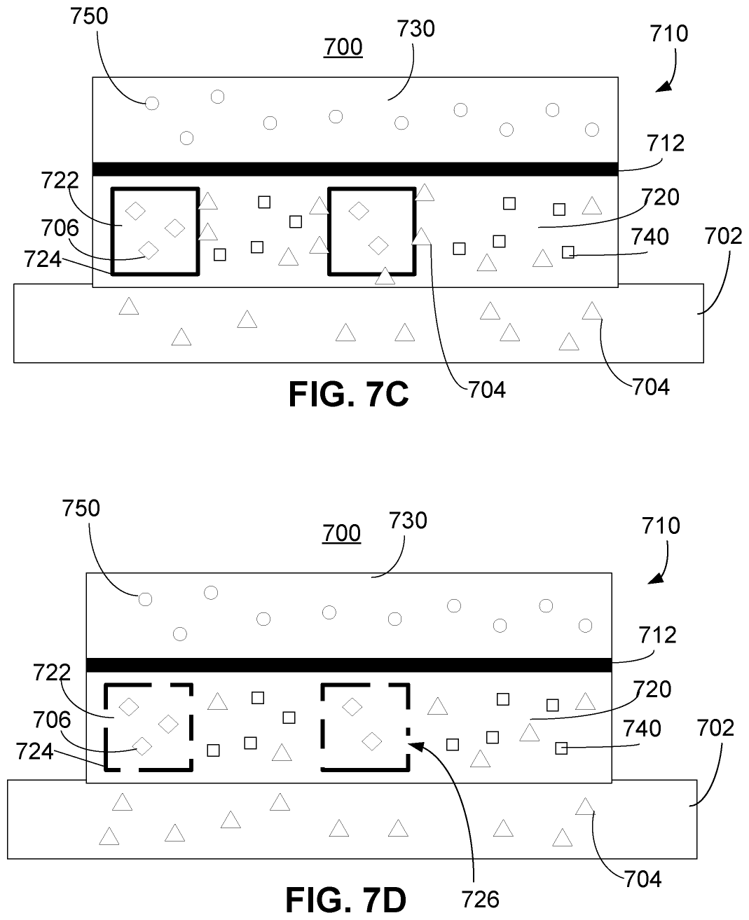

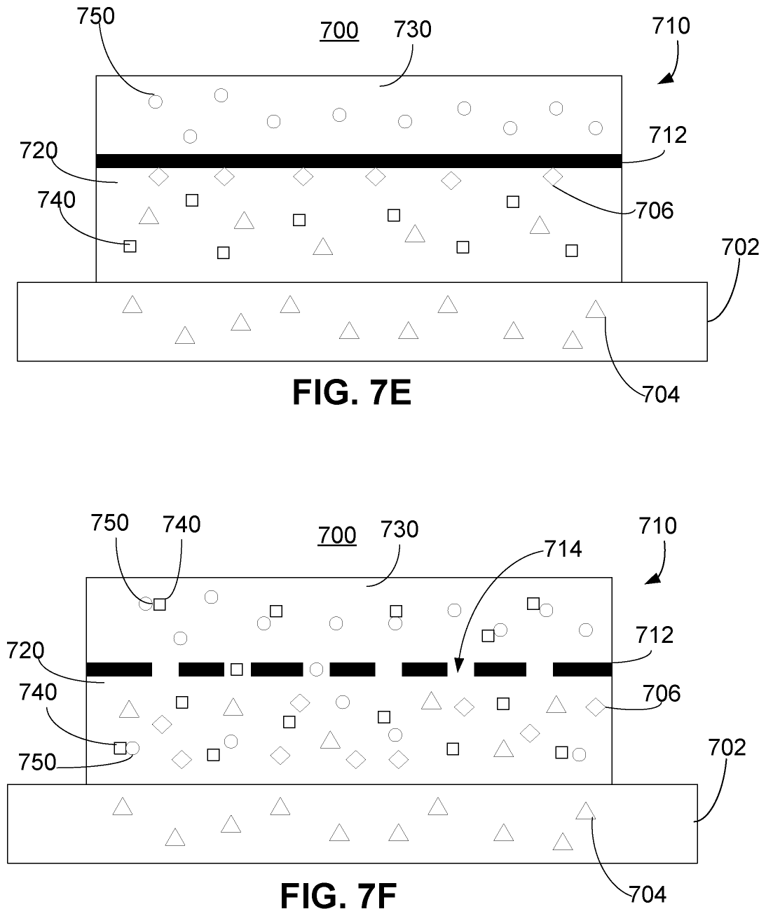

The primary agent need not act directly on the barrier to achieve the permeable state. The primary agent can activate a secondary agent to achieve the permeable state. The secondary agent can comprise, for example, an enzyme capable of cleaving the barrier, a pore forming agent, a dissolution agent, a tearing agent, or a vehicle capable of carrying the fluorophore, the quencher, or both across the barrier. The primary agent can activate the secondary agent by converting the secondary agent from a sequestered state to a free state. For example, the secondary agent can be attached to a tether, contained in a vesicle, or both in the sequestered state. The primary agent can cleave the tether, affect a conformational change in the tether, or both to release the secondary agent from the tether. In another example, the primary agent can release the secondary agent from the vesicle. In yet another example, the primary agent can activate the secondary agent by enzymatically modifying the secondary agent, conformationally altering the secondary agent, or both. Examples of embodiments that employ a secondary agent are shown in FIGS. 6A-7F.

FIG. 6A depicts a side schematic view of a system 600 comprising device 610 before application to a substrate 602 comprising primary agent 604. A barrier 612 in an impermeable state separates fluorophores 640 in an absorption matrix 620 from quenchers 650 in a container 630. Secondary agents 606 are anchored by tethers 608 in absorption matrix 620 to prevent them from affecting barrier 612. FIG. 6B depicts device 610 in FIG. 6A after application to substrate 602 and before absorption of primary agent 604. FIG. 6C depicts device 610 after absorbing primary agent 604 with primary agent 604 affecting tethers 608. FIG. 6D depicts device 610 after secondary agent 606 has been released by primary agent 604. After being cleaved or otherwise affected, the tethers can remain free but partially attached to the absorption matrix or secondary agent. Alternatively, the tethers can essentially disintegrate. FIG. 6E depicts barrier 612 in an impermeable state being affected by secondary agent 606. FIG. 6F depicts fluorophores 640 interacting with quenchers 650 after crossing barrier 612 in the permeable state. Holes 614 in barrier 612 represent the permeable state.

FIG. 7A depicts a side schematic view of a system 700 comprising device 710 before application to a substrate 702 comprising primary agent 704. Vesicles 722 are defined by a membrane 724 in an impermeable state containing secondary agents 706 to prevent them from affecting the barrier. A barrier 712 separates fluorophores 740 in an absorption matrix 720 from quenchers 750 in container 730. FIG. 7B depicts device 710 in FIG. 7A after application to substrate 702 and before absorption of primary agent 704. FIG. 7C depicts device 710 after absorbing primary agent 704 with the primary agent 704 affecting membranes 724 of vesicles 722. FIG. 7D depicts device 710 after secondary agent 706 has been released from vesicles 722 by primary agent 704. Holes 726 represent membrane 724 in a permeable state. FIG. 7E depicts barrier 712 in an impermeable state being affected by secondary agent 706. FIG. 7F depicts fluorophores 740 interacting with quenchers 750 after crossing barrier 712 in the permeable state. Holes 714 represent barrier 712 in a permeable state.

While the above description of FIGS. 6A-7F is presented with only a single species of primary agent 604 or 704, secondary agent 606 or 706, barrier 612 or 712, fluorophore 640 or 740, and quencher 750 or 750, respectively, in some implementations a plurality of different species may be provided. For example, the device 610 or 710 may include multiple active areas laterally displaced from one another, each corresponding to a different wound biomarker (e.g., MMPs, TIMPs, pH indicators, and the like) and each with a unique fluorescence emission wavelength. Concentrations and relative levels of each biomarker can be read out and analyzed by, for example, an AI algorithm.

To implement the multiple active areas, the container can comprise one or more compartments. For example, the container can comprise a first compartment and a second compartment. Both compartments can be adjacent to the barrier. The fluorophore can be a first fluorophore and the system can further comprise a second fluorophore. The first and second fluorophores can differ in excitation wavelength, emission wavelength, or both. The first compartment can comprise the first fluorophore, and the second compartment can comprise the second fluorophore. The absorption matrix can comprise the quencher in the impermeable state. The quencher can be configured to quench the first fluorophore or the second fluorophore. The quencher can be configured to quench both the first fluorophore and the second fluorophore.

The primary agent can be a first primary agent and the absorption matrix can be further configured to receive a second primary agent from the substrate. The barrier can comprise a first region separating the first compartment from the absorption matrix in the impermeable state, and a second region separating the second compartment from the absorption matrix in the impermeable state. The first primary agent can be configured to convert the first region from the impermeable state to the permeable state. The second primary agent can be configured to convert the second region from the impermeable state to the permeable state.

The quencher can be a first quencher, and the system can further comprise a second quencher. The first quencher can be configured to quench the first fluorophore. The second quencher can be configured to quench the second fluorophore. The absorption matrix can comprise a first chamber adjacent to the first region and a second chamber adjacent to the second region. The first chamber can comprise the first quencher in the impermeable state. The second chamber can comprise the second quencher in the impermeable state. The presence of chambers, containers, or both can give the device a quilted appearance. A chamber of the absorption matrix can be formulated to absorb a particular type or types of primary agents of interest.

The container can comprise a first compartment and a second compartment. Both compartments can be adjacent to the barrier. The quencher can be a first quencher, and the system can further comprise a second quencher. The first and second quenchers can differ in their abilities to quench the fluorophore. The first compartment can comprise the first quencher. The second compartment can comprise the second quencher. The absorption matrix can comprise the fluorophore in the impermeable state. The first quencher or the second quencher can be configured to quench the fluorophore. Both the first quencher and the second quencher can be configured to quench the fluorophore. The primary agent can be a first primary agent, and the absorption matrix can be further configured to receive a second primary agent from the substrate. The barrier can comprise a first region separating the first compartment from the absorption matrix in the impermeable state, and a second region separating the second compartment from the absorption matrix in the impermeable state. The first primary agent can be configured to convert the first region from the impermeable state to the permeable state. The second primary agent can be configured to convert the second region from the impermeable state to the permeable state. The fluorophore can be a first fluorophore, the system further comprising a second fluorophore. The first quencher can be configured to quench the first fluorophore. The second quencher can be configured to quench the second fluorophore. The absorption matrix can comprise a first chamber adjacent to the first region and a second chamber adjacent to the second region. The first chamber can comprise the first fluorophore in the impermeable state. The second chamber can comprise the second fluorophore in the impermeable state.

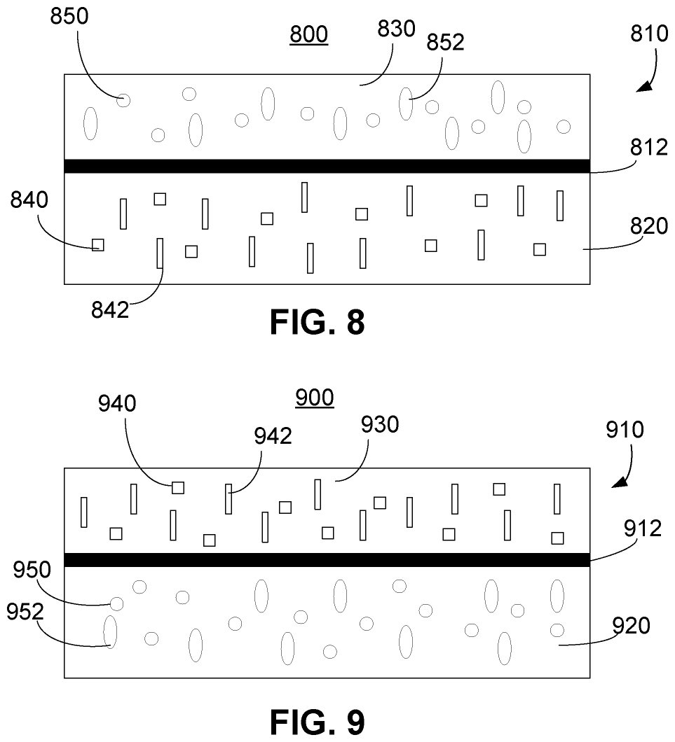

FIGS. 8 and 9 depict examples of embodiments comprising multiple fluorophores and multiple quenchers. FIG. 8 depicts a side schematic view of a system 800 comprising a device 810 before application to a substrate 802 comprising a primary agent 804. A barrier 812 in an impermeable state separates first and second fluorophores 840, 842 in an absorption matrix 820 from first and second quenchers 850, 852 in a container 830. Device 810 is analogous to device 110 shown in FIG. 1A apart from the addition of second fluorophores 842 and second quenchers 852. FIG. 9 depicts a side schematic view of a system 900 comprising a device 910 before application to a substrate 902 comprising a primary agent 904. A barrier 912 in an impermeable state separates first and second quenchers 950, 952 in an absorption matrix 920 separated from first and second fluorophores 940, 942 in a container 930. Device 910 is analogous to device 210 shown in FIG. 2A apart from the addition of second fluorophore 942 and second quencher 952. The disclosure also encompasses embodiments in which a first fluorophore and a second quencher are on one side of a barrier and a second fluorophore and first quencher are on the other side of the barrier. There can be any desired number of types of fluorophores and quenchers. The number of fluorophore types can be the same or differ from the number of quenchers. Multiple fluorophore types can interact with a single type of quencher. Multiple quenchers can interact with a single type of fluorophore.

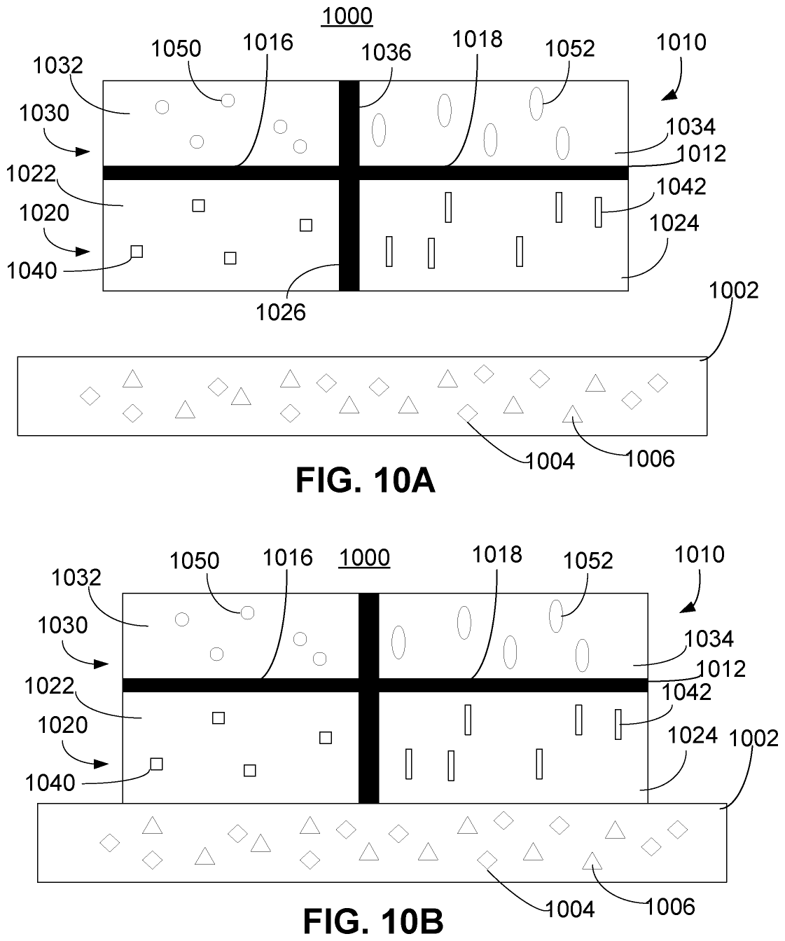

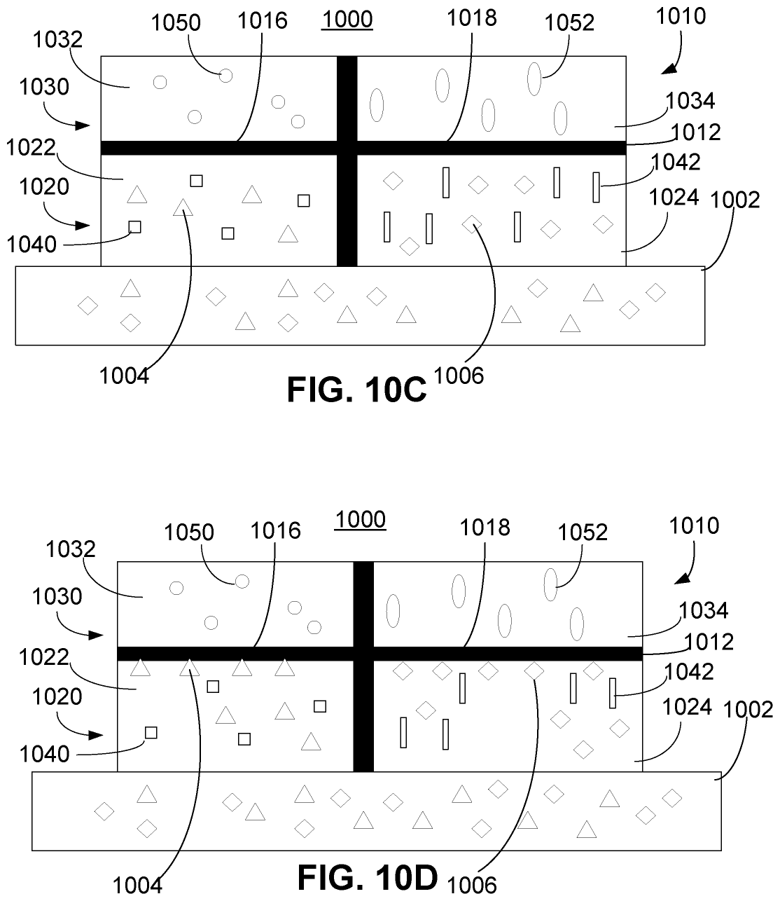

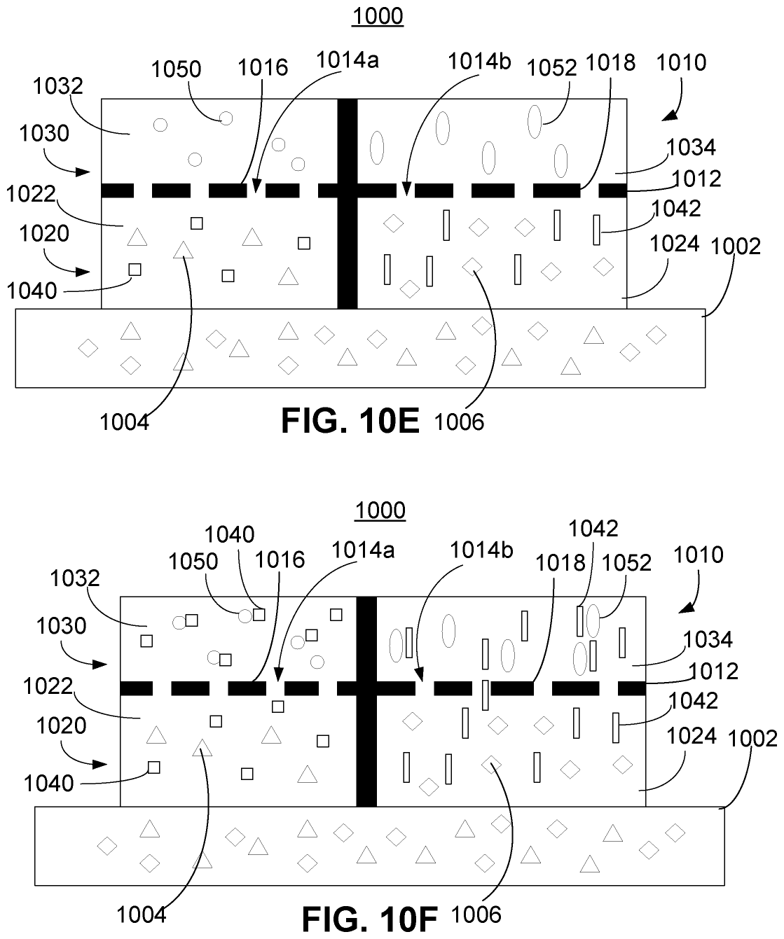

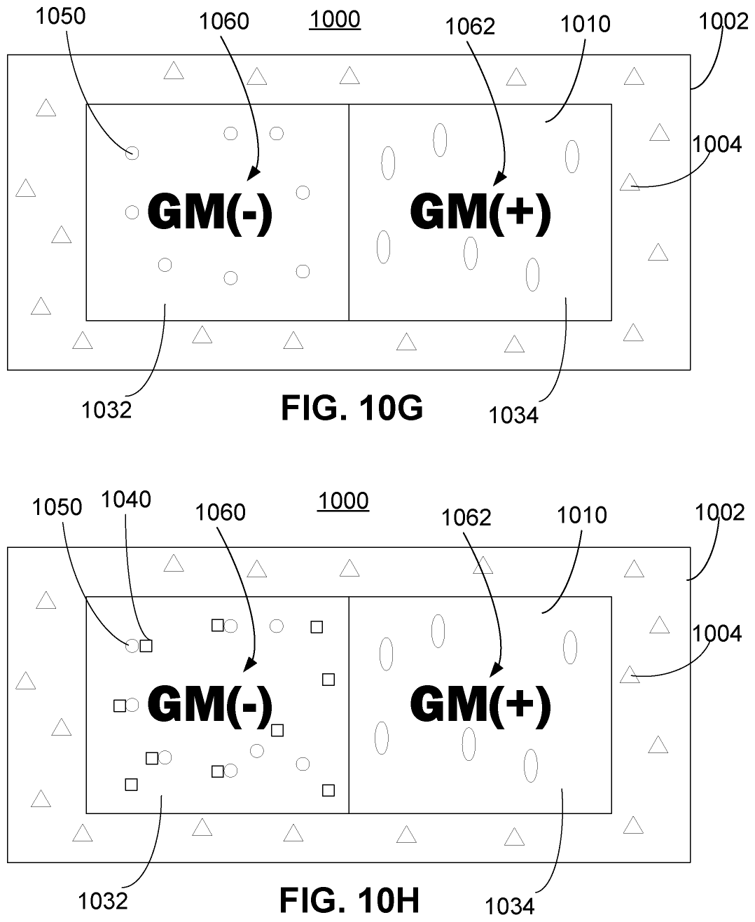

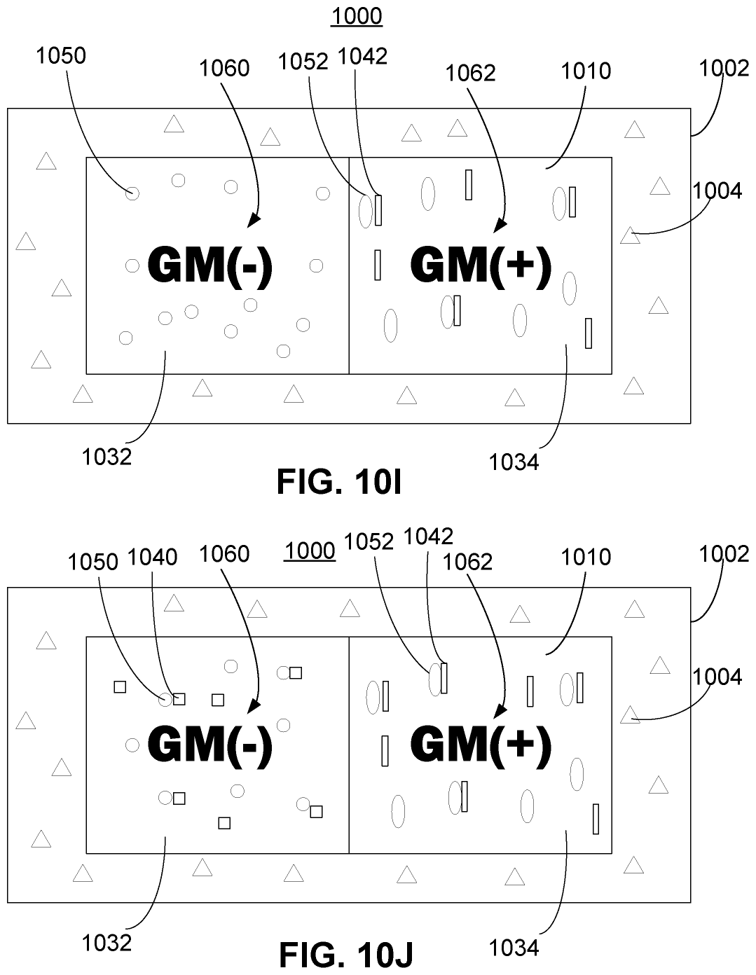

FIGS. 10A-10J depict an example of embodiments comprising multiple fluorophores and multiple quenchers with further compartmentalization of the absorption matrix and the container. These figures also depict an example of embodiments in which the system is configured to detect multiple types of primary agents. Apart from the multiple fluorophores, quenchers, primary agents, and further compartmentalization, these figures are analogous to FIGS. 1A-1H. Accordingly, FIG. 10A depicts a side schematic view of a system 1000 comprising a device 1010 before application to a substrate 1002 comprising first primary agent 1004 and second primary agent 1006. A barrier 1012 in an impermeable state separates first and second fluorophores 1040, 1042 in respective chambers 1022, 1024 of an absorption matrix 1020 from first and second quenchers 1052, 1054 in respective compartments 1032, 1034 of a container 1030. Chambers 1022, 1024 are separated by a chamber divider 1026. Chambers 1022, 1024 can be configured with a material composition that favors the absorption of first primary agent 1004 in the former and second primary agent 1006 in the latter. Compartments 1032, 1034 are separated by a compartment divider 1036.

FIG. 10B depicts device 1010 in FIG. 10A after application to substrate 1002 and before absorption of first and second primary agents 1004, 1006. FIG. 10C depicts device 1010 after absorbing first and second primary agents 1004, 1006. FIG. 10D depicts first and second primary agents 1004, 1006 affecting respective first and second regions 1016, 1018 of barrier 112. FIG. 10E depicts barrier 1012 in a permeable state after being affected by first and second primary agents 1004, 1006. Holes 1014a, 1014b represent first and second regions 1016, 1018 of barrier 1012 in a permeable state. FIG. 10F depicts first and second fluorophores 1040, 1042 interacting with the first and second quenchers 1050, 1052, respectively, after crossing barrier 1012 into respective compartments 1032, 1034 of container 1030 in the permeable state of regions 1016, 1018.

FIGS. 10G-10J are analogous to FIGS. 1G and 1H, but include two instead of one type of graphic indicia. “GM(−)” and “GM(+)” (for Gram negative and Gram positive bacteria) are only examples of indicia, as is “BAC” in FIGS. 1G and 1H. The graphic indicia can refer to, for example, the identity of the primary agent, the type of cell that produces the primary agent, a secondary agent, a recommended therapeutic regimen, or the like. 10G is a plan view corresponding to device 1010 depicted in FIGS. 10A-10D in which the first and second graphic indicia 1060, 1062 are visible but not highlighted. FIG. 10H is a plan view of device 1010 in which first graphic indicia 1060 are highlighted indicating the presence of first primary agent 1004. FIG. 10I is a plan view of device 1010 in which second graphic indicia 1062 are highlighted indicating the presence of second primary agent 1006. FIG. 10J is a plan view corresponding to device 1010 depicted in FIG. 10F in which both first and second graphic indicia 1060, 1062 are highlighted by fluorescence emitted from device 1010 indicating the presence of both first and second primary agents 1004, 1006. In other implementations, at least some of the graphic indicia 1060 and/or 1062 may initially be invisible and may become visible as a result of energy transfer from fluorophores to quenchers. Moreover, the graphic indicia 1060 and/or 1062 may initially be partly visible and partly invisible and become fully visible as a result of the energy transfer; for example, to change from a minus sign to a plus sign.

In accordance with the present disclosure, a fluorescence reporting system is provided. The system can comprise a fluorophore, a linker, a quencher, and an absorption matrix. The quencher can be configured to quench the fluorophore in a quenching state. The linker can be configured to maintain the quenching state. The absorption matrix can comprise the fluorophore, the quencher, and the linker. The absorption matrix can be configured to receive a primary agent from a substrate. The primary agent can be configured to establish a non-quenching state.

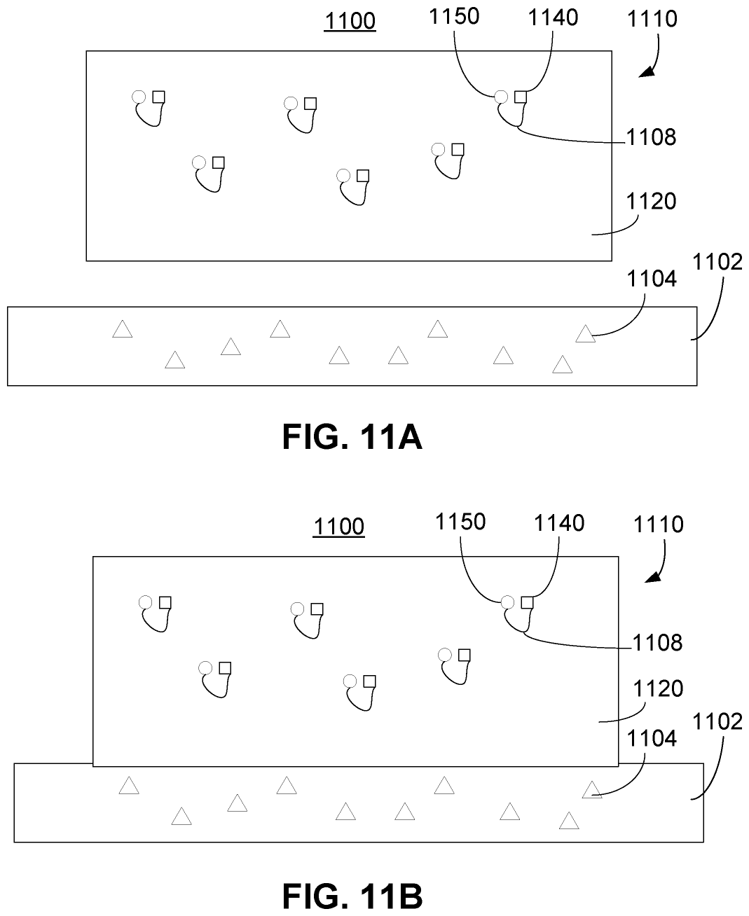

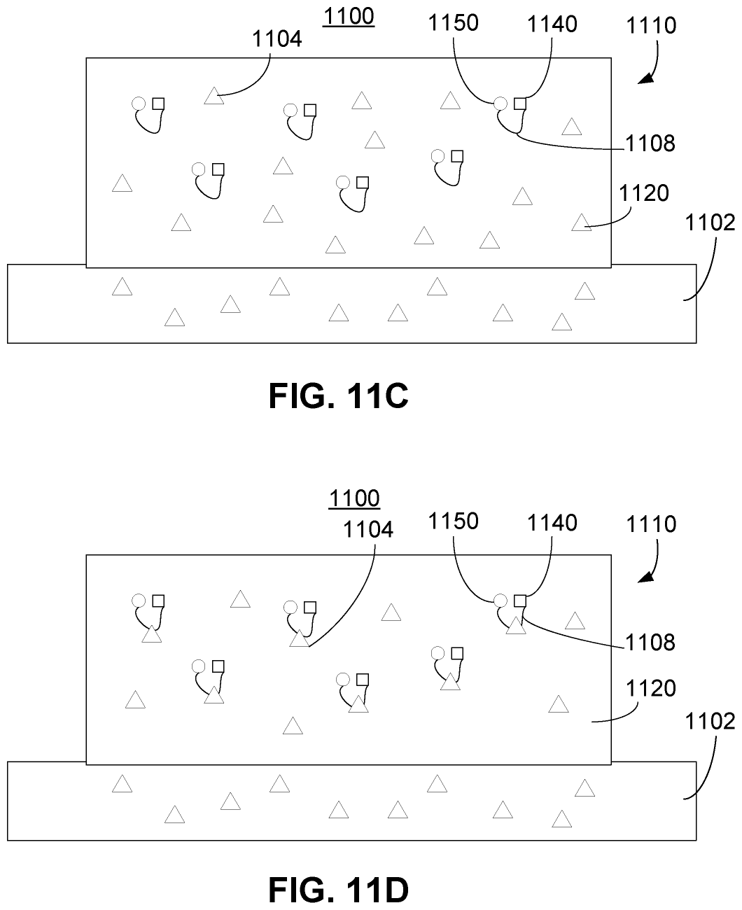

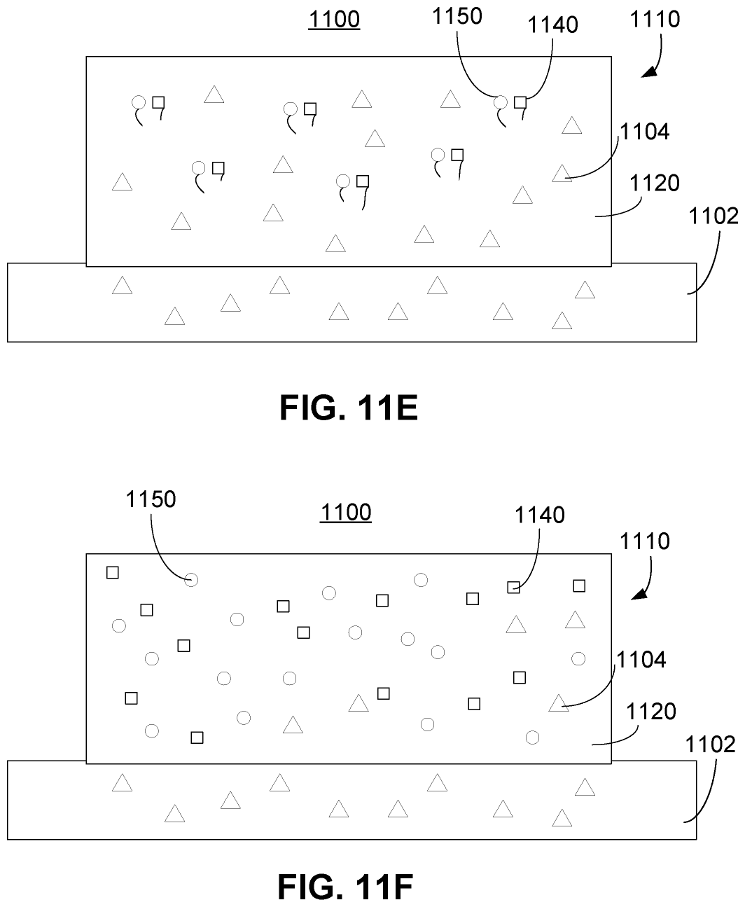

The primary agent can establish the non-quenching state using one or more mechanisms. For example, the primary agent can bind to the linker to establish the non-quenching state. The primary agent can cleave the linker to establish the non-quenching state. The primary agent chemically can modify the linker to establish the non-quenching state. The primary agent can establish the non-quenching state by binding to a stabilizer, the stabilizer binding the linker in the quenching state. The primary agent can comprise an enzyme. The primary agent can activate a secondary agent to establish the non-quenching state. The secondary agent can bind to the linker, cleave the linker, remove a stabilizer molecule from the linker, or chemically modify the linker, or any combination thereof. FIGS. 11A-13D show examples of embodiments in which the effective distance of between the fluorophores and quenchers is increased to establish the non-quenching state.

FIG. 11A depicts a side schematic view of a system 1100 comprising a device 1110 before application to a substrate 1102 comprising primary agent 1104. Fluorophores 1140 are bound to quenchers 1150 through respective linkers 1108. Linkers 1108 permit fluorophores 1140 and quenchers 1150 to achieve sufficiently proximity for energy transfer to occur. FIG. 11B depicts device 1110 in FIG. 11A after application to substrate 1102 and before absorption of primary agent 1104. FIG. 11C depicts device 1110 after primary agent 1104 has been absorbed. FIG. 11D depicts the system after primary agent 1104 has bound to linkers 1008. FIG. 11E depicts device 1110 after primary agent 1104 has cleaved linkers 1008. FIG. 11F depicts device 1110 after previously linked fluorophores 1140 and quenchers 1150 have dispersed and no longer, on average have a sufficient proximity to one another for energy transfer to occur.

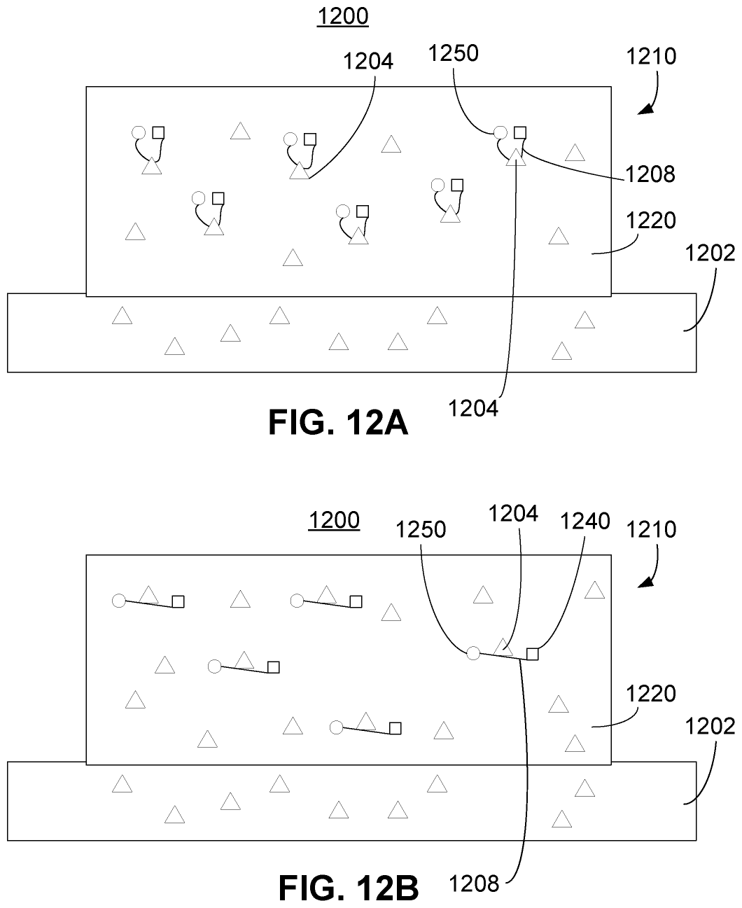

FIG. 12A depicts a side schematic view of a system 1200 comprising a device 1210 with fluorophores 1240 bound to quenchers 1250 through respective linkers 1208. The linkers permit fluorophores 1240 and quenchers 1250 to achieve sufficient proximity for energy transfer to occur in a first configuration. Device 1210 has been applied to a substrate 1202 and has absorbed primary agent 1204. Primary agent 1204 has begun to affect linkers 1208. FIG. 12B depicts device 1210 after primary agent 1204 has converted linkers 1208 to a second configuration such that fluorophores 1240 and quenchers 1250 are no longer sufficiently close for energy transfer to occur. Alternatively, the primary agent can first activate a secondary agent that performs the conversion. The conversion to the second configuration can be temporary or permanent. For example, the second configuration can be maintained if the primary agent remains bound to linkers 1208, and loss of the primary agent can then result in conversion back to the first configuration. Alternatively, the second configuration can be maintained even after primary agent 1204 has disengaged from linkers 1208. For example, primary agent can enzymatic modify linkers 1208 so that the second configuration is maintained. Another agent can then bind linkers 1208 and reestablish the first configuration.

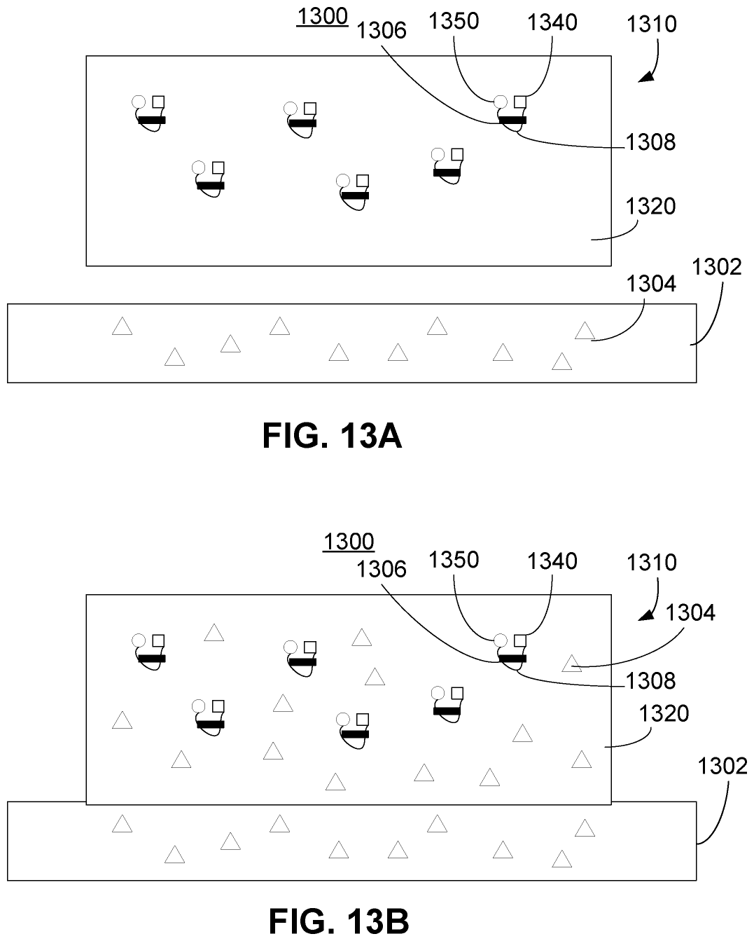

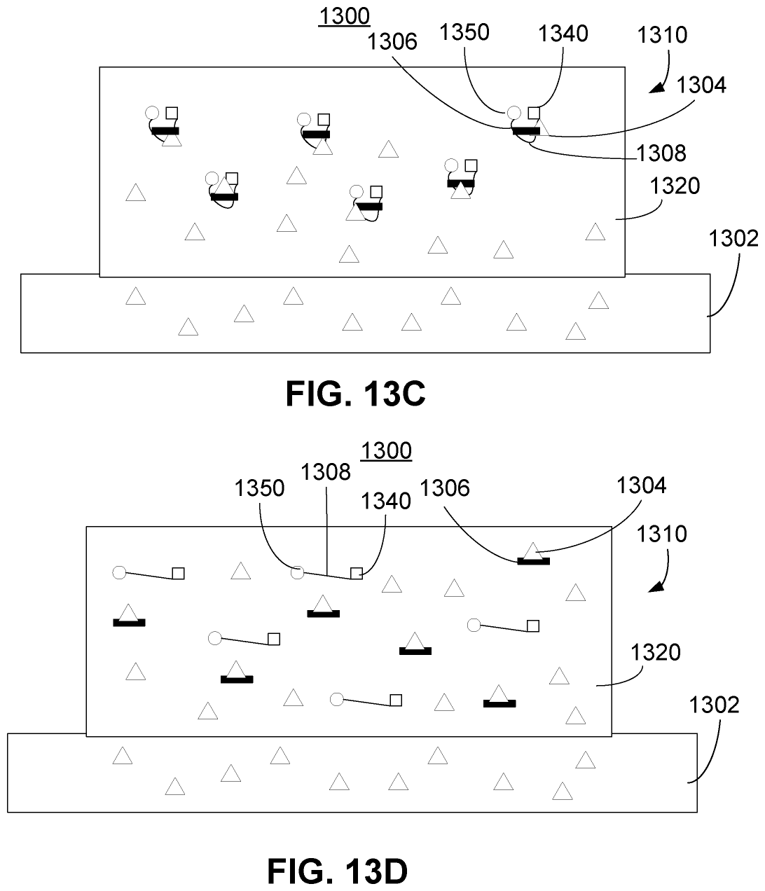

FIG. 13A depicts a side schematic view of a system 1300 comprising a device 1310 before application to a substrate 1302 comprising primary agent 1304. Fluorophores 1340 are bound to quenchers 1350 through respective linkers 1308. Linkers 1308 permit fluorophores 1340 and quenchers 1350 to achieve sufficiently proximity for energy transfer in a first configuration maintained by a secondary agent 1306. FIG. 13B depicts device 1310 in FIG. 13A after application to substrate 1302 and after absorption of primary agent 1304 from substrate 1302. FIG. 13C depicts device 1310 after primary agent 1304 has bound to secondary agent 1306. FIG. 13D depicts device 1310 after primary agent 1304 has converted linkers 1308 to a second configuration by removing secondary agent 1306 from linkers 1308 such that fluorophores 1340 and quenchers 1350 are no longer sufficiently close for energy transfer to occur.

The fluorophore can be a first fluorophore, and the system can further comprise a second fluorophore. The first and second fluorophores can differ in excitation wavelength, emission wavelength, or both. The linker can be a first linker, the system further comprising a second linker. The first and second linkers can be configured to maintain the quenching state. The quencher can be a first quencher, the system further comprising a second quencher. The first quencher ca be configured to quench the first fluorophore, and the second quencher can be configured to quench the second fluorophore. The first linker can link the first fluorophore and the first quencher. The second linker can link the second fluorophore and the second quencher. The primary agent can be a first primary agent, the system further comprising a second primary agent.

The absorption matrix can comprise the first fluorophore, the second fluorophore, the first quencher, the second quencher, the first linker, and the second linker. The absorption matrix can be configured to receive both the first primary agent and the second primary agent from the substrate. The first primary agent can be configured to establish the non-quenching state for the first fluorophore and the first quencher. The second primary agent can be configured to establish the non-quenching state for the second fluorophore and the second quencher. The combined quenching states for the first and second fluorophore can exhibit a first color or an absence of color. The quenching state for the first fluorophore combined with the non-quenching state for the second fluorophore can exhibit a second color. The quenching state for the second fluorophore combined with the non-quenching state for the first fluorophore can exhibit a third color. Combined non-quenching states for the first and second fluorophores can exhibit a fourth color or an absence of color. Color as used herein, unless otherwise specified, can refer to a single wavelength or a plurality of wavelength of electromagnetic radiation comprising visible light, light outside the visible spectrum, or both.

In accordance with the present disclosure, a fluorescence reporting system is provided. The system can comprise a fluorophore, a quencher, and an absorption matrix. The quencher can be configured to quench the fluorophore in a quenching state. The absorption matrix can comprise the fluorophore and the quencher. The absorption matrix can be configured to receive a primary agent from a substrate. The primary agent can be configured to establish the quenching state by decreasing an average distance between the fluorophore and the quencher.

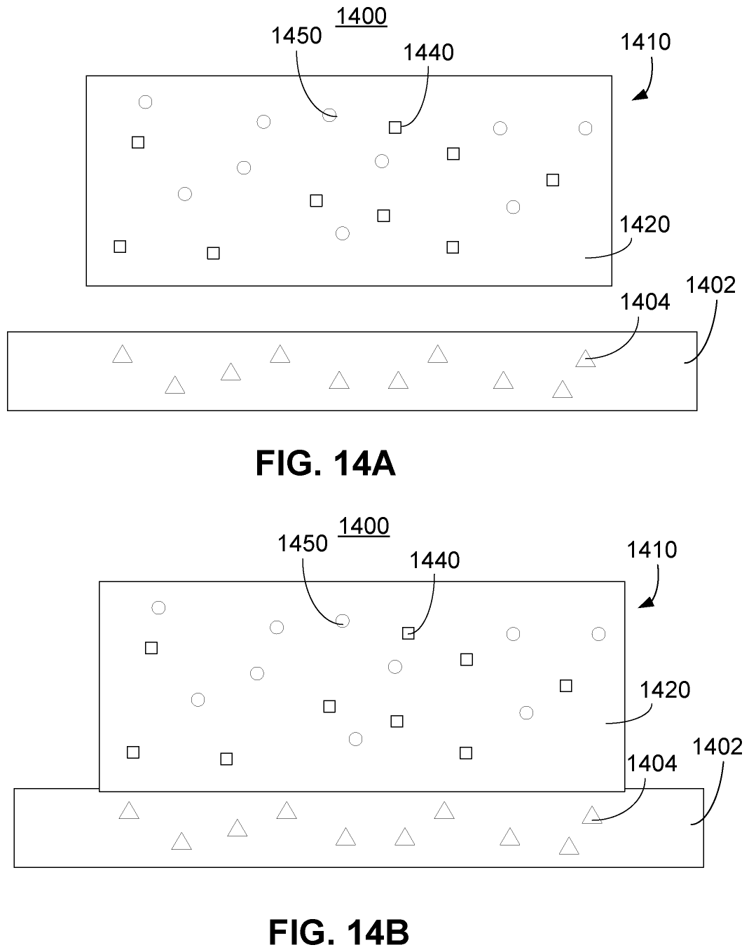

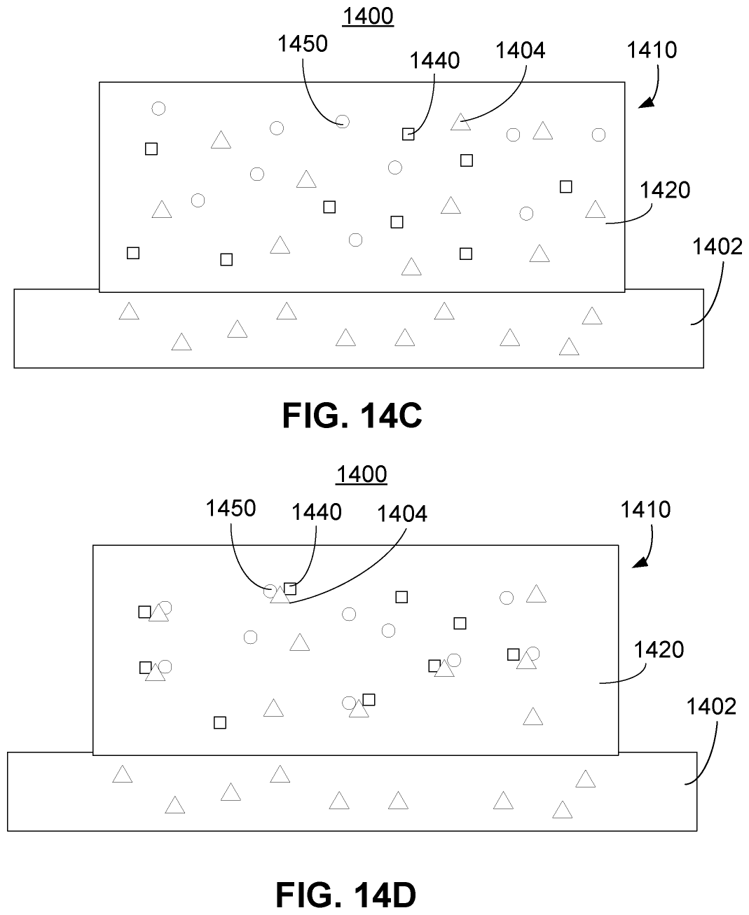

The primary agent can decrease the average distance between the fluorophore and the quencher directly or indirectly. The primary agent can bind the fluorophore and the quencher to decrease the average distance. The primary agent can alter a property of the absorption matrix to decrease the average distance. For example, the property can comprise at least one of viscosity, degree of cross-linking, density, solubility, polarity, ionic strength, pH, hydrophilicity, hydrophobicity, water content, and temperature. The primary agent can comprise, for example, an enzyme. The primary agent can activate, for example, a secondary agent to establish the quenching state or inactivate the secondary agent to terminate the quenching state. The secondary agent can, for example, bind the fluorophore and the quencher and/or modify a property of the absorption matrix. The establishment of the quenching state can change a color of the system. FIGS. 14A-14D show an example of embodiments in which a primary agent directly decreases the proximity between the fluorophores and quenchers by binding to both.

FIG. 14A depicts a side schematic view of system 1400 comprising a device 1410 before application to a substrate 1402 comprising primary agent 1404. Fluorophores 1440 and quenchers 1450 are freely dispersed in an absorption matrix 1420 without sufficient proximity to one another for energy transfer to occur. FIG. 14B depicts device 1410 in FIG. 14A after application to substrate 1402 and before absorption of primary agent 1404. FIG. 14C depicts device 1410 after primary agent 1404 has been absorbed. FIG. 14D depicts device 1410 after primary agent 1404 has bound both fluorophores 1440 and quenchers 1450 permitting them to achieve sufficiently proximity for energy transfer to occur. Alternatively, primary agent 1404 can first activate a secondary agent that then binds fluorophores 1440 and quenchers 1450.

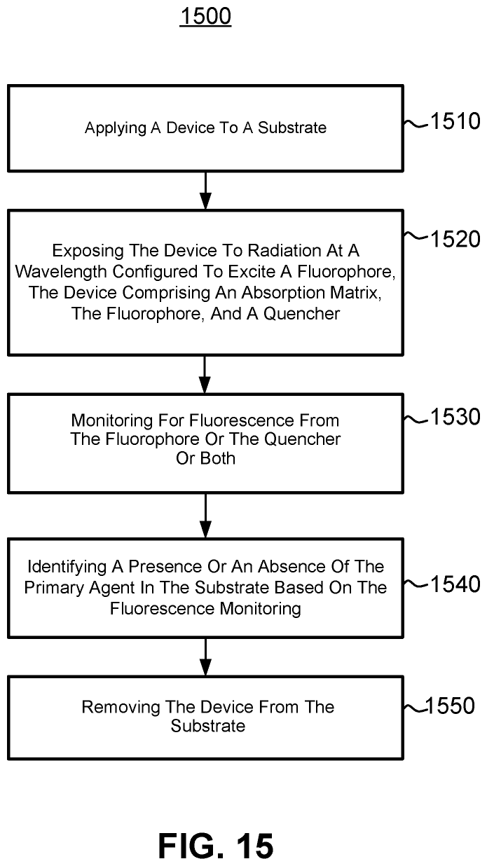

In accordance with the present disclosure, a method of characterizing a substrate is provided. FIG. 15 shows an example of a method in accordance with the present disclosure. FIG. 15 is a flow chart depicting steps of a method 1500 for characterizing the status of a substrate. The method can comprise one or more of the following steps. A device on, or previously adjacent to, a substrate can be exposed to radiation at a wavelength configured to excite a fluorophore (1520). The device can comprise, for example, any system described herein. For example, the device can comprise an absorption matrix, the fluorophore, and a quencher. The quencher can be configured to quench the fluorophore. The absorption matrix can be configured to receive a primary agent from the substrate. The primary agent can be configured to affect the quenching of the fluorophore by the quencher. Fluorescence from the fluorophore or the quencher or both can be monitored (1530). A presence or an absence of the primary agent in the substrate can be identified based on the fluorescence monitoring (1540). The method can comprise applying the device to the substrate (1510), removing the device from the substrate (1550), or both.

The method can comprise detecting fluorescence. The fluorescence detected can indicate the presence or absence of the primary agent in the substrate. The monitoring can be performed at a first time and repeated at a second time. The monitoring can comprise detecting a change in fluorescence at the second time relative to the first time. The change can indicate a change in a concentration of the primary agent in the substrate.

The substrate comprises a living tissue, or a dead tissue, or both. The substrate can comprise a consumable. The device can be a consumable packaging. The absorption matrix can be in fluid communication with the consumable. The method can comprise discarding the consumable if fluorescence is detected or not detected as indicative of contamination, infection, spoilage, or both. That is, identification of the primary agent can be indicative of contamination, infection, spoilage, or both.

The substrate can comprise a wound. The device can comprise a bandage. The absorption matrix can be in fluid communication with the wound. The primary agent can comprise a molecule indicative of bacteria. The bacteria can comprise, for example, a pathogenic bacterium, a toxin-producing bacterium, a tissue damaging bacterium, or a bacterium associated with a negative sensory profile of a consumable, or any combination thereof. The primary agent can comprise a bacterial molecule, or a host molecule, or both. The target area can comprise at least one wound. The wound can be any type of wound. For example, the wound can comprise an abrasion, a laceration, a puncture, an avulsion, a bruise, a contusion, a bite, a burn, a rash, frostbite, a boil, a mole, a pimple, a cyst, an ulcer (such as a diabetic ulcer), a bed sore, or any combination thereof. The wound can be deep or superficial. A wound can be external on or in the skin, or internal on or in an internal membrane. The wound can comprise the epidermis, the dermis, or the hypodermis, or any combination thereof. The wound can be infected or uninfected. An infected wound can be infected with any kind, number, or combination of organisms. The infection can comprise one or more bacteria, one or more fungi, one or more protozoa, or one or more viruses, or any combination thereof. The organism can be monocellular, multicellular, or non-cellular.

As noted above, devices as described herein may be used to make measurements over time to document changes in wound and/or healthy tissue parameters. For example, the monitoring can be performed at a first time and repeated at a second time such that the monitoring at the first time is indicative of a presence of the bacteria, the monitoring at the second time is indicative of an absence of the bacteria. The method can comprise removing the device from the substrate at or after the second time when no bacteria is detected or no bacteria above a chosen threshold.

The primary agent identified can comprise a molecule indicative of a cancer, for example, a melanoma. The substrate can comprise a tissue suspected of cancer, a precancerous tissue, a benign cancerous tissue, a malignant cancerous tissue, or a metastatic cancerous tissue, or any combination thereof. The method can comprise identifying the presence of the primary agent and removing the device from the substrate after the primary agent is identified. The method can comprise identifying the presence of the primary agent and performing a cancer treatment to the substrate. The cancer treatment can comprise surgery, chemotherapy, or radiation, or any combination thereof. The device applied and then removed can be a first device, and the method can further comprises applying a second device to the substrate after performing the cancer treatment.

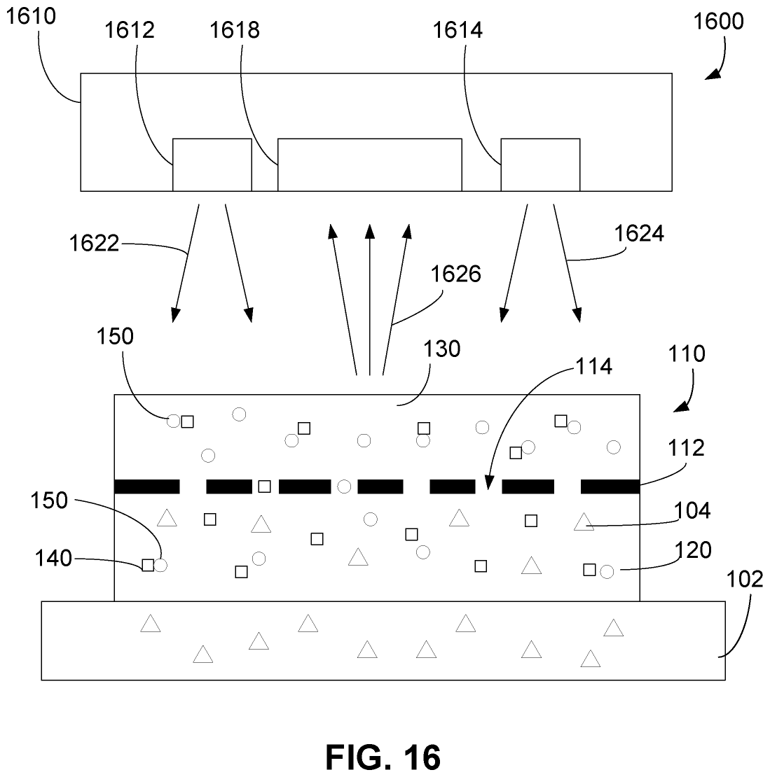

Monitoring over time and/or the non-invasive diagnosis of wounds may be accomplished through the use of a multispectral imaging device. FIG. 16 depicts an example of embodiments implementing such monitoring. FIG. 16 depicts a side schematic view of a system 1600 comprising a device 110 (i.e., the device 110 illustrated in FIGS. 1A-1H) and a multispectral imager 1610. However, the multispectral imager 1610 is not limited to use only with the device 110, and may instead be used with any device described above (e.g., any of the devices 210, 310, 410, 510, 610, 710, 810, 910, 1010, 1110, 1210, 1310, and/or 1410 illustrated in FIGS. 2A-14D. For clarity of explanation, descriptions of the internal components of the device 110 are not repeated here.

The multispectral imager 1610 includes light emitting elements 1612 and 1614 configured to emit radiation 1622 and 1624 toward the device 110, and a light detecting element 1618 configured to receive responsive radiation 1626 from the device 110. While only two light emitting elements and one light detecting element are shown in FIG. 16, in practical implementations additional elements may be included. For example, the multispectral imager 1610 may include one or more additional light detecting elements laterally displaced from the light detecting element 1618 to permit stereoscopic imaging and/or imaging of different wavelength ranges. Imaging of different wavelength ranges may additionally or alternatively be accomplished with the use of a variable filter and/or a selectable filtering mechanism including a plurality of selectable filters. Additionally, the multispectral imager 1610 may include additional light emitting elements to permit excitation in different wavelength ranges, white light imaging, IR rangefinder, and the like.

The light emitting elements 1612 and 1614 may be light emitting diodes (LEDs). Radiation 1622 and radiation 1624 may correspond to the same wavelength or wavelength range or may correspond to different wavelengths or wavelength ranges. The wavelengths of emitted radiation may lie in the UV, visible, or IR ranges. In one example, radiation 1622 and radiation 1624 may both correspond to a wavelength of 405 nm±15 nm. In another example, radiation 1622 may correspond to a wavelength of 405 nm±15 nm and radiation 1624 may correspond to white light.

The light detecting element 1618 may be any device configured to convert radiation 1626 into an electric signal. For example, the light detecting element 1618 may be or include a solid-state image sensor, such as a Complementary Metal-Oxide Semiconductor (CMOS) image sensor, a Charge-Coupled Device (CCD), and the like. The multispectral imager 1610 may be equipped with circuits and other components to permit image processing or other operations, such as processors, memories, driving circuits, input/output (I/O) interfaces, user interfaces (UIs), displays, communication circuitry, and the like.

In use, the device 110 may be applied to a target area, such as a wound, by a practitioner at a healthcare facility. Subsequently, the multispectral imager 1610 may be used to emit radiation 1622 and radiation 1624 both corresponding to a wavelength of 405 nm±15 nm or each corresponding to a different excitation wavelength. The device 110 may be configured with multiple different fluorescence probes (e.g., different fluorophores 140 and quenchers 150) in the reporter layer, each probe corresponding to a different biomarker. Each fluorescence probe provides a different wavelength component of radiation 1626 back to the multispectral imager 1610, which is captured by the light detecting element 1618. The multispectral imager 1610 may then provide real-time or other readout corresponding to the radiation 1626, in the form of images, spectra, graphs, charts, text indicators, and the like. The readout may indicate the status of the wound at the time of imaging, such as the protease level, acidity, fibroblast activity, etc., and therefore provide information regarding the healing status of the wound, such as an indication of infection, biofilm presence, likelihood of healing, etc. Operation of the multispectral imager 1610 may be conducted by a patient (e.g., at the patient's home) and the readout may be sent to a doctor at another location (e.g., at the healthcare facility). The operation of the multispectral imager 1610 may be performed repeatedly at predetermined or prescribed intervals to thereby provide a series of readouts for monitoring of the course of healing of the wound. After a number of repeated operations, the patient may then return to the healthcare facility where the device 110 may be replaced.

The readout may include the use of a lookup table (LUT) and AI algorithm. For example, the AI algorithm may analyze bacteria fluorescence images, determine wound area or other parameters, and predict wound healing using well-characterized wound biomarkers to increase the accuracy of the diagnosis. This may also be used to indicate the efficacy of treatment; for example, if wound debridement has reduced bacterial load, then bacteria-secreted biomarkers (e.g., protease) levels would decrease over time and the series of readouts would show this.

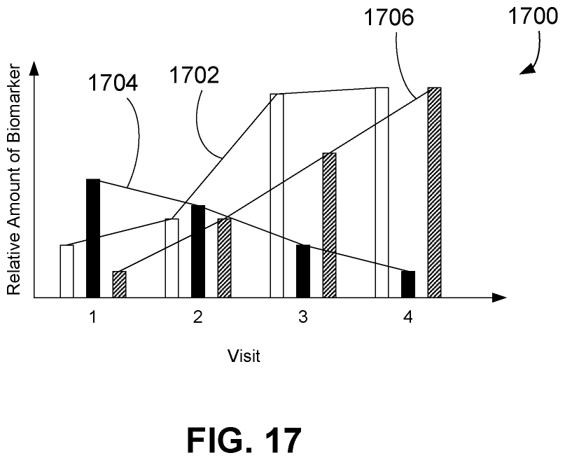

FIG. 17 depicts an exemplary series of readouts 1700, each in bar graph form. In FIG. 17, the vertical axis corresponds to a relative amount of biomarker present in arbitrary units and the horizontal axis corresponds to the visit at which the biomarkers are measured (e.g., the multispectral imaging analysis is conducted). The unfilled box corresponds to MMP, and the change thereof from visit to visit is depicted as trendline 1702. The solid box corresponds to TIMP, and the change thereof from visit to visit is depicted as trendline 1704. The hatched box corresponds to pH, and the change thereof from visit to visit is depicted as trendline 1706. In the depiction of FIG. 17, wound MMP increased, wound TIMP decreased, and pH increased over four visits. The AI algorithm may analyze this series of readouts and determine a diagnosis: a chronic wound that is infected with biofilm.

The present disclosure can include any combination of these various features or embodiments above and/or below as set forth in sentences and/or paragraphs. Any combination of disclosed features herein is considered part of the present disclosure and no limitation is intended with respect to combinable features.

The present disclosure includes the following aspects/embodiments/features in any order and/or in any combination.
1. A fluorescence reporting system comprising: a fluorophore; a quencher configured to quench the fluorophore; an absorption matrix configured to receive a primary agent from a substrate; a barrier, the primary agent converting the barrier from an impermeable state to a permeable state, the barrier impermeable to the fluorophore and the quencher in the impermeable state; and a container separated from the absorption matrix by the barrier, wherein: the absorption matrix comprises the fluorophore and the container comprises the quencher in the impermeable state, or the absorption matrix comprises the quencher and the container comprises the fluorophore in the impermeable state.
2. The system of any preceding or following aspect/embodiment/feature, wherein the quencher is fluorescent.
3. The system of any preceding or following aspect/embodiment/feature, wherein the fluorophore transfers energy to the quencher via fluorescence resonance energy transfer (FRET).
4. The system of any preceding or following aspect/embodiment/feature, wherein the quencher is not fluorescent.
5. The system of any preceding or following aspect/embodiment/feature, wherein quenching of the fluorophore by the quencher results in a change in emitted radiation in the system.
6. The system of any preceding or following aspect/embodiment/feature, wherein the change in emitted radiation comprises a change in color.
7. The system of any preceding or following aspect/embodiment/feature, wherein the absorption matrix comprises a hydrogel.
8. The system of any preceding or following aspect/embodiment/feature, wherein the barrier comprises a membrane.
9. The system of any preceding or following aspect/embodiment/feature, further comprising a first solution comprising the fluorophore and a second solution comprising the quencher, wherein: the absorption matrix comprises the first solution and the container comprises the second solution in the impermeable state, or the absorption matrix comprises the second solution and the container comprises the first solution in the impermeable state.
10. The system of any preceding or following aspect/embodiment/feature, wherein the first and second solutions are miscible.
11. The system of any preceding or following aspect/embodiment/feature, wherein the system further comprises: a first layer comprising the absorption matrix; a second layer comprising the barrier; and a third layer comprising the container.
12. The system of any preceding or following aspect/embodiment/feature, wherein the container is a vesicle surrounded by the absorption matrix.
13. The system of any preceding or following aspect/embodiment/feature, wherein the absorption matrix comprises a plurality of containers, the plurality comprising the container.
14. The system of any preceding or following aspect/embodiment/feature, wherein the system further comprises a housing holding the absorption matrix, the barrier, and the container.
15. The system of any preceding or following aspect/embodiment/feature, further comprising an attachment on the housing configured to attach the housing to the substrate.
16. The system of any preceding or following aspect/embodiment/feature, wherein the attachment comprises an adhesive.
17. The system of any preceding or following aspect/embodiment/feature, wherein the housing is transparent, translucent, or both.
18. The system of any preceding or following aspect/embodiment/feature, wherein the housing is flexible.
19. The system of any preceding or following aspect/embodiment/feature, wherein the housing is a bandage.
20. The system of any preceding or following aspect/embodiment/feature, wherein the substrate comprises a wound.
21. The system of any preceding or following aspect/embodiment/feature, wherein the housing comprises a graphic feature comprising text, a number, a letter, a symbol, or a pattern, or any combination thereof indicating a state of the system.
22. The system of any preceding or following aspect/embodiment/feature, wherein the graphic feature is configured to be highlighted by fluorescence from the fluorophore, the quencher, or both.
23. The system of any preceding or following aspect/embodiment/feature, wherein the housing further comprises a light source configured to excite the fluorophore.
24. The system of any preceding or following aspect/embodiment/feature, wherein the housing further comprises a detector configured to detect fluorescence emitted by the fluorophore, fluorescence emitted by the quencher, or both.
25. The system of any preceding or following aspect/embodiment/feature, wherein the substrate comprises a living tissue.
26. The system of any preceding or following aspect/embodiment/feature, wherein the substrate comprises a dead tissue.
27. The system of any preceding or following aspect/embodiment/feature, wherein the substrate comprises at least one of a wound, a lesion, an abscess, a surgical or post-surgical site, an incision point, a treatment device that provides physical access into a patient body, or combinations thereof.
28. The system of any preceding or following aspect/embodiment/feature, wherein the container further comprises a substrate-affecting agent.
29. The system of any preceding or following aspect/embodiment/feature, wherein the substrate-affecting agent comprises a therapeutic agent.
30. The system of any preceding or following aspect/embodiment/feature, wherein the therapeutic agent comprises an antibiotic, an analgesic, or both.
31. The system of any preceding or following aspect/embodiment/feature, wherein the primary agent comprises an enzyme.
32. The system of any preceding or following aspect/embodiment/feature, wherein the enzyme comprises a pathogenic enzyme, a host enzyme, or both.
33. The system of any preceding or following aspect/embodiment/feature, wherein the enzyme cleaves the barrier to achieve the permeable state.
34. The system of any preceding or following aspect/embodiment/feature, wherein the enzyme comprises a matrix metalloprotease.
35. The system of any preceding or following aspect/embodiment/feature, wherein the primary agent activates a secondary agent to achieve the permeable state.
36. The system of any preceding or following aspect/embodiment/feature, wherein the secondary agent comprises an enzyme capable of cleaving the barrier, a pore forming agent, a dissolution agent, a tearing agent, or a vehicle capable of carrying the fluorophore, the quencher, or both across the barrier.
37. The system of any preceding or following aspect/embodiment/feature, wherein the primary agent activates the secondary agent by converting the secondary agent from a sequestered state to a free state.
38. The system of any preceding or following aspect/embodiment/feature, wherein the secondary agent is attached to a tether, contained in a vesicle, or both in the sequestered state.
39. The system of any preceding or following aspect/embodiment/feature, wherein the primary agent cleaves the tether, affects a conformational change in the tether, or both to release the secondary agent from the tether.
40. The system of any preceding or following aspect/embodiment/feature, wherein the primary agent releases the secondary agent from the vesicle.
41. The system of any preceding or following aspect/embodiment/feature, wherein the primary agent activates the secondary agent by enzymatically modifying the secondary agent, conformationally altering the secondary agent, or both.
42. The system of any preceding or following aspect/embodiment/feature, wherein: the container comprises a first compartment and a second compartment, both adjacent to the barrier; the fluorophore is a first fluorophore and the system further comprises a second fluorophore, the first and second fluorophores different in excitation wavelength, emission wavelength, or both; the first compartment comprises the first fluorophore; the second compartment comprises the second fluorophore; and the absorption matrix comprises the quencher in the impermeable state.
43. The system of any preceding or following aspect/embodiment/feature, wherein the quencher is configured to quench the first fluorophore or the second fluorophore.
44. The system of any preceding or following aspect/embodiment/feature, wherein the quencher is configured to quench both the first fluorophore and the second fluorophore.
45. The system of any preceding or following aspect/embodiment/feature, wherein: the primary agent is a first primary agent and the absorption matrix is further configured to receive a second primary agent from the substrate; the barrier comprises a first region separating the first compartment from the absorption matrix in the impermeable state, and a second region separating the second compartment from the absorption matrix in the impermeable state; the first primary agent is configured to convert the first region from the impermeable state to the permeable state; and the second primary agent is configured to convert the second region from the impermeable state to the permeable state.
46. The system of any preceding or following aspect/embodiment/feature, wherein: the quencher is a first quencher, the system further comprising a second quencher; the first quencher is configured to quench the first fluorophore; the second quencher is configured to quench the second fluorophore; the absorption matrix comprises a first chamber adjacent to the first region and a second chamber adjacent to the second region; the first chamber comprises the first quencher in the impermeable state; and the second chamber comprises the second quencher in the impermeable state.
47. The system of any preceding or following aspect/embodiment/feature, wherein: the container comprises a first compartment and a second compartment, both adjacent to the barrier; the quencher is a first quencher and the system further comprises a second quencher, the first and second quenchers differing in their abilities to quench the fluorophore; the first compartment comprises the first quencher; and the second compartment comprises the second quencher; and the absorption matrix comprises the fluorophore in the impermeable state.
48. The system of any preceding or following aspect/embodiment/feature, wherein the first quencher or the second quencher is configured to quench the fluorophore.
49. The system of any preceding or following aspect/embodiment/feature, wherein both the first quencher and the second quencher are configured to quench the fluorophore.
50. The system of any preceding or following aspect/embodiment/feature, wherein: the primary agent is a first primary agent and the absorption matrix is further configured to receive a second primary agent from the substrate; the barrier comprises a first region separating the first compartment from the absorption matrix in the impermeable state, and a second region separating the second compartment from the absorption matrix in the impermeable state; the first primary agent is configured to convert the first region from the impermeable state to the permeable state; and the second primary agent is configured to convert the second region from the impermeable state to the permeable state.
51. The system of any preceding or following aspect/embodiment/feature, wherein: the fluorophore is a first fluorophore, the system further comprising a second fluorophore; the first quencher is configured to quench the first fluorophore; the second quencher is configured to quench the second fluorophore; the absorption matrix comprises a first chamber adjacent to the first region and a second chamber adjacent to the second region; the first chamber comprises the first fluorophore in the impermeable state; and the second chamber comprises the second fluorophore in the impermeable state.
52. The system of any preceding or following aspect/embodiment/feature, wherein the fluorophore exhibits temperature dependence.
53. A fluorescence reporting system comprising: a fluorophore; a linker; a quencher configured to quench the fluorophore in a quenching state, the linker configured to maintain the quenching state; and an absorption matrix comprising the fluorophore, the quencher, and the linker, the absorption matrix configured to receive a primary agent from a substrate, the primary agent configured to establish a non-quenching state.
54. The system of any preceding or following aspect/embodiment/feature, wherein the primary agent binds to the linker to establish the non-quenching state.
55. The system of any preceding or following aspect/embodiment/feature, wherein the primary agent cleaves the linker to establish the non-quenching state.
56. The system of any preceding or following aspect/embodiment/feature, wherein the primary agent chemically modifies the linker to establish the non-quenching state.
57. The system of any preceding or following aspect/embodiment/feature, wherein the primary agent establishes the non-quenching state by binding to a stabilizer, the stabilizer binding the linker in the quenching state.
58. The system of any preceding or following aspect/embodiment/feature, wherein the primary agent comprises an enzyme.
59. The system of any preceding or following aspect/embodiment/feature, wherein the primary agent activates a secondary agent to establish the non-quenching state.
60. The system of any preceding or following aspect/embodiment/feature, wherein the secondary agent binds to the linker, cleaves the linker, removes a stabilizer molecule from the linker, or chemically modifies the linker, or any combination thereof.
61. The system of any preceding or following aspect/embodiment/feature, wherein the system further comprises a housing holding the absorption matrix.
62. The system of any preceding or following aspect/embodiment/feature, further comprising an attachment on the housing configured to attach the housing to the substrate.
63. The system of any preceding or following aspect/embodiment/feature, wherein the housing is a bandage.
64. The system of any preceding or following aspect/embodiment/feature, wherein establishment of the non-quenching state changes a color of the system.
65. The system of any preceding or following aspect/embodiment/feature, the fluorophore is a first fluorophore, the system further comprising a second fluorophore, the first and second fluorophores different in excitation wavelength, emission wavelength, or both; the linker is a first linker, the system further comprising a second linker, the first and second linkers configured to maintain the quenching state; the quencher is a first quencher, the system further comprising a second quencher, the first quencher configured to quench the first fluorophore, and the second quencher configured to quench the second fluorophore; the first linker linking the first fluorophore and the first quencher; the second linker linking the second fluorophore and the second quencher; the primary agent is a first primary agent, the system further comprising a second primary agent; the absorption matrix comprising the first fluorophore, the second fluorophore, the first quencher, the second quencher, the first linker, and the second linker, the absorption matrix configured to receive both the first primary agent and the second primary agent from the substrate; the first primary agent configured to establish the non-quenching state for the first fluorophore and the first quencher; and the second primary agent configured to establish the non-quenching state for the second fluorophore and the second quencher.
66. The system of any preceding or following aspect/embodiment/feature, wherein: combined quenching states for the first and second fluorophore exhibits a first color; the quenching state for the first fluorophore combined with the non-quenching state for the second fluorophore exhibits a second color; the quenching state for the second fluorophore combined with the non-quenching state for the first fluorophore exhibits a third color; combined non-quenching states for the first and second fluorophores exhibits a fourth color.
67. A fluorescence reporting system comprising: a fluorophore; a quencher configured to quench the fluorophore in a quenching state; and an absorption matrix comprising the fluorophore and the quencher, the absorption matrix configured to receive a primary agent from a substrate, the primary agent configured to establish the quenching state by decreasing an average distance between the fluorophore and the quencher to establish the quenching state.
68. The system of any preceding or following aspect/embodiment/feature, wherein the primary agent binds the fluorophore and the quencher.
69. The system of any preceding or following aspect/embodiment/feature, wherein the primary agent alters a property of the absorption matrix.
70. The system of any preceding or following aspect/embodiment/feature, wherein the property comprises at least one of viscosity, degree of cross-linking, density, solubility, polarity, ionic strength, pH, hydrophilicity, hydrophobicity, water content, and temperature.
71. The system of any preceding or following aspect/embodiment/feature, wherein the primary agent comprises an enzyme.
72. The system of any preceding or following aspect/embodiment/feature, wherein the primary agent activates a secondary agent to establish the quenching state or inactivates the secondary agent to terminate the quenching state.
73. The system of any preceding or following aspect/embodiment/feature, wherein the secondary agent binds the fluorophore and the quencher.
74. The system of any preceding or following aspect/embodiment/feature, wherein the system further comprises a housing holding the absorption matrix.
75. The system of any preceding or following aspect/embodiment/feature, further comprising an attachment on the housing configured to attach the housing to the substrate.
76. The system of any preceding or following aspect/embodiment/feature, wherein the housing is a bandage.
77. The system of any preceding or following aspect/embodiment/feature, wherein establishment of the quenching state changes a color of the system.
78. A method of characterizing a substrate, the method comprising: exposing a device on a substrate to radiation at a wavelength configured to excite a fluorophore, the device comprising an absorption matrix, the fluorophore, and a quencher, the quencher configured to quench the fluorophore, the absorption matrix configured to receive a primary agent from the substrate, the primary agent configured to affect the quenching of the fluorophore by the quencher; monitoring for fluorescence from the fluorophore or the quencher or both; identifying a presence or an absence of the primary agent in the substrate based on the fluorescence monitoring.
79. The method of any preceding or following aspect/embodiment/feature, further comprising applying the device to the substrate.
80. The method of any preceding or following aspect/embodiment/feature, further comprising removing the device from the substrate.
81. The method of any preceding or following aspect/embodiment/feature, further comprising detecting fluorescence.
82. The method of any preceding or following aspect/embodiment/feature, wherein the fluorescence detected indicates the presence or absence of the primary agent in the substrate.
83. The method of any preceding or following aspect/embodiment/feature, wherein the monitoring is performed at a first time and repeated at a second time.
84. The method of any preceding or following aspect/embodiment/feature, wherein the monitoring comprises detecting a change in fluorescence at the second time relative to the first time.
85. The method of any preceding or following aspect/embodiment/feature, wherein the change indicates a change in a concentration of the primary agent in the substrate.
86. The method of any preceding or following aspect/embodiment/feature, wherein the substrate comprises a living tissue, or a dead tissue, a wound, a lesion, an abscess, a surgical or post-surgical site, an incision point, a treatment device that provides physical access into a patient body, or combinations thereof.
87. The method of any preceding or following aspect/embodiment/feature, wherein the device is a consumable packaging.
88. The method of any preceding or following aspect/embodiment/feature, wherein the substrate comprises a consumable.
89. The method of any preceding or following aspect/embodiment/feature, wherein the device is a bandage.
90. The method of any preceding or following aspect/embodiment/feature, wherein the substrate comprises a wound.
91. The method of any preceding or following aspect/embodiment/feature, wherein the absorption matrix is in fluid communication with the wound.
92. The method of any preceding or following aspect/embodiment/feature, wherein the primary agent comprises a molecule indicative of bacteria.
93. The method of any preceding or following aspect/embodiment/feature, wherein the bacteria comprises a pathogenic bacterium, a toxin-producing bacterium, a tissue damaging bacterium, or a bacterium associated with a negative sensory profile of a consumable, or any combination thereof.
94. The method of any preceding or following aspect/embodiment/feature, wherein the primary agent comprises a bacterial molecule, or a host molecule, or both.
95. The method of any preceding or following aspect/embodiment/feature, wherein the monitoring is performed at a first time and repeated at a second time, the monitoring at the first time is indicative of a presence of the bacteria, the monitoring at the second time is indicative of an absence of the bacteria.
96. The method of any preceding or following aspect/embodiment/feature, further comprising removing the device from the substrate at or after the second time.
97. The method of any preceding or following aspect/embodiment/feature, the substrate comprises a consumable, the absorption matrix is in fluid communication with the consumable, the presence of the primary agent is identified, and the method further comprises discarding the consumable.
98. The method of any preceding or following aspect/embodiment/feature, wherein the primary agent comprises a molecule indicative of a cancer.
99. The method of any preceding or following aspect/embodiment/feature, wherein the substrate comprises a tissue suspected of cancer, a precancerous tissue, a benign cancerous tissue, a malignant cancerous tissue, or a metastatic cancerous tissue, or any combination thereof.
100. The method of any preceding or following aspect/embodiment/feature, wherein the presence of the primary agent is identified, and the method further comprises: removing the device from the substrate after the primary agent is identified; and performing a cancer treatment to the substrate.
101. The method of any preceding or following aspect/embodiment/feature, wherein the cancer treatment comprises surgery, chemotherapy, or radiation, or any combination thereof.
102. The method of any preceding or following aspect/embodiment/feature, wherein the device is a first device, and the method further comprises applying a second device to the substrate after performing the cancer treatment.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

1. A fluorescence reporting system comprising:
a fluorophore;
a quencher configured to quench the fluorophore;
an absorption matrix configured to receive a primary agent from a substrate;
a barrier, the primary agent converting the barrier from an impermeable state to a permeable state, the barrier impermeable to the fluorophore and the quencher in the impermeable state; and
a container separated from the absorption matrix by the barrier, wherein:
the absorption matrix comprises the fluorophore and the container comprises the quencher in the impermeable state, or
the absorption matrix comprises the quencher and the container comprises the fluorophore in the impermeable state.

2. The system of claim 1, wherein the quencher is fluorescent.

3. The system of claim 2, wherein the fluorophore transfers energy to the quencher via fluorescence resonance energy transfer (FRET).

4. (canceled)

5. The system of claim 1, wherein quenching of the fluorophore by the quencher results in a change in emitted radiation in the system.

6. The system of claim 5, wherein the change in emitted radiation comprises a change in color.

7. The system of claim 1, wherein the absorption matrix comprises a hydrogel.

8. The system of claim 1, wherein the barrier comprises a membrane.

9. The system of claim 1, further comprising a first solution comprising the fluorophore and a second solution comprising the quencher, wherein:
the absorption matrix comprises the first solution and the container comprises the second solution in the impermeable state, or
the absorption matrix comprises the second solution and the container comprises the first solution in the impermeable state.

10. The system of claim 9, wherein the first and second solutions are miscible.

11. The system of claim 1, wherein the system further comprises:
a first layer comprising the absorption matrix;
a second layer comprising the barrier; and
a third layer comprising the container.

12.-13. (canceled)

14. The system of claim 1, wherein the system further comprises a housing holding the absorption matrix, the barrier, and the container.

15. The system of claim 14, further comprising an attachment on the housing configured to attach the housing to the substrate.

16.-18. (canceled)

19. The system of claim 14, wherein the housing is a bandage.

20. The system of claim 19, wherein the substrate comprises a wound.

21. The system of claim 19, wherein the housing comprises a graphic feature comprising text, a number, a letter, a symbol, or a pattern, or any combination thereof indicating a state of the system.

22. The system of claim 21, wherein the graphic feature is configured to be highlighted by fluorescence from the fluorophore, the quencher, or both.

23. The system of claim 14, wherein the housing further comprises a light source configured to excite the fluorophore.

24. The system of claim 14, wherein the housing further comprises a detector configured to detect fluorescence emitted by the fluorophore, fluorescence emitted by the quencher, or both.

25.-31. (canceled)

32. The system of claim 1, wherein the primary agent comprises a pathogenic enzyme, a host enzyme, or both.

33. The system of claim 1, wherein the enzyme cleaves the barrier to achieve the permeable state.

34.-41. (canceled)

42. The system of claim 1, wherein:
the container comprises a first compartment and a second compartment, both adjacent to the barrier;
the fluorophore is a first fluorophore and the system further comprises a second fluorophore, the first and second fluorophores different in excitation wavelength, emission wavelength, or both;
the first compartment comprises the first fluorophore;
the second compartment comprises the second fluorophore; and
the absorption matrix comprises the quencher in the impermeable state.

43.-52. (canceled)

53. A fluorescence reporting system comprising:
a fluorophore;
a linker;
a quencher configured to quench the fluorophore in a quenching state, the linker configured to maintain the quenching state; and
an absorption matrix comprising the fluorophore, the quencher, and the linker, the absorption matrix configured to receive a primary agent from a substrate, the primary agent configured to establish a non-quenching state.

54.-64. (canceled)

65. The system of claim 53, wherein:
the fluorophore is a first fluorophore, the system further comprising a second fluorophore, the first and second fluorophores different in excitation wavelength, emission wavelength, or both;
the linker is a first linker, the system further comprising a second linker, the first and second linkers configured to maintain the quenching state;
the quencher is a first quencher, the system further comprising a second quencher, the first quencher configured to quench the first fluorophore, and the second quencher configured to quench the second fluorophore;
the first linker linking the first fluorophore and the first quencher
the second linker linking the second fluorophore and the second quencher;
the primary agent is a first primary agent, the system further comprising a second primary agent;
the absorption matrix comprising the first fluorophore, the second fluorophore, the first quencher, the second quencher, the first linker, and the second linker, the absorption matrix configured to receive both the first primary agent and the second primary agent from the substrate;
the first primary agent configured to establish the non-quenching state for the first fluorophore and the first quencher; and
the second primary agent configured to establish the non-quenching state for the second fluorophore and the second quencher.

66. The system of claim 65, wherein:
combined quenching states for the first and second fluorophore exhibits a first color;
the quenching state for the first fluorophore combined with the non-quenching state for the second fluorophore exhibits a second color;
the quenching state for the second fluorophore combined with the non-quenching state for the first fluorophore exhibits a third color;
combined non-quenching states for the first and second fluorophores exhibits a fourth color.

67.-102. (canceled)
